(19) United States
(12) Patent Application Publication
(10) Pub. No.: US 2007/0042016 A1
(43) Pub. Date: Feb. 22, 2007

(54) Methods and Systems for Treating Injured Cardiac Tissue

(75) Inventors: Asha S. Nayak, Menlo Park, CA (US); James R. Keogh, Maplewood, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: MEDTRONIC VASCULAR, INC., Santa Rosa, CA (US)

(21) Appl. No.: 11/426,211

(22) Filed: Jun. 23, 2006

Related U.S. Application Data
(60) Provisional application No. 60/693,749, filed on Jun. 23, 2005.
(60) Provisional application No. 60/743,686, filed on Mar. 23, 2006.

(51) Int. Cl. A61N 1/30; A61F 2/00; A61B 1/32; A61B 17/08

(52) U.S. Cl. 424/423; 600/37; 600/205; 606/213; 604/20

(57) ABSTRACT

Methods and systems are disclosed for treating injury to cardiac tissue by delivering a composition which provides structural support to the cardiac tissue. The composition helps to prevent chamber remodeling by providing structural reinforcement of the tissue or structural reinforcement of the tissue combined with biological therapy. The structurally reinforcing composition can thicken the wall of a heart, or act to prevent further thinning and thereby provide resistance against further remodeling. A number of compositions are disclosed, including multi-component substances such as autologous platelet gel, and other substances. The compositions disclosed can contain additives to augment/enhance the desired effects of the injection.

### CROSS REFERENCE TO RELATED APPLICATIONS

The present application claims priority under 35 U.S.C. §119(e) to U.S. Provisional Patent Application Nos. 60/693,749 filed Jun. 23, 2005 and 60/743,686 filed Mar. 23, 2006.

### FIELD OF THE INVENTION

The present invention relates generally to systems and methods for treating injured, ischemic, or infarcted tissue. Specifically, the present invention discloses methods of providing structural reinforcement of the tissue alone, and structural reinforcement of the tissue combined with biological support to injured, ischemic, or infarcted cardiac tissue, thus reducing or eliminating remodeling of heart chambers that can occur in such tissue.

### BACKGROUND OF THE INVENTION

The human heart wall consists of an inner layer of simple squamous epithelium, referred to as the endocardium, overlying a variably thick heart muscle or myocardium and is enveloped within a multi-layer tissue structure referred to as the pericardium. The innermost layer of the pericardium, referred to as the visceral pericardium or epicardium, covers the myocardium. The epicardium reflects outward at the origin of the aortic arch to form an outer tissue layer, referred to as the parietal pericardium, which is spaced from and forms an enclosed sac extending around the visceral pericardium of the ventricles and atria. An outermost layer of the pericardium, referred to as the fibrous pericardium, attaches the parietal pericardium to the sternum, the great vessels and the diaphragm so that the heart is confined within the middle mediastinum. Normally, the visceral pericardium and parietal pericardium lie in close contact with each other and are separated only by a thin layer of a serous pericardial fluid that enables friction free movement of the heart within the sac. The space between the visceral and parietal pericardia is referred to as the pericardial space. In common parlance, the visceral pericardium is usually referred to as the epicardium, and epicardium will be used hereafter. Similarly, the parietal pericardium is usually referred to as the pericardium, and pericardium will be used hereafter in reference to parietal pericardium.

Heart disease, including myocardial infarction, is a leading cause of death and disability in human beings, particularly in the western world, most particularly among males. A variety of heart diseases can progress to heart failure by a common mechanism called remodeling. With remodeling, cardiac function progressively deteriorates, often leading to clinical heart failure and associated symptoms. Heart disease can in turn impair other physiological systems. Each year over 1.1 million Americans have a myocardial infarction (MI). Myocardial infarction can result in an acute depression in ventricular function and expansion of the infarcted tissue under stress. This triggers a cascading sequence of myocellular events known as remodeling. In many cases, this progressive myocardial infarct expansion and remodeling leads to deterioration in ventricular function and heart failure. Such ischemic cardiomyopathy is the leading cause of heart failure in the United States. It is the objective of the present invention to prevent heart failure in high-risk patients who are at risk of suffering or who have suffered from an ischemic or other injurious event likely to lead to remodeling and heart failure.

A stenosed or blocked coronary artery is one example of heart disease. A completely or substantially blocked coronary artery can cause immediate, intermediate term, and/or long-term adverse effects. In the immediate term, a myocardial infarction (MI) can occur when a coronary artery becomes occluded and can no longer supply blood to the myocardial tissue, thereby resulting in myocardial cell death. When a myocardial infarction occurs, the myocardial tissue that is no longer receiving adequate blood flow dies and is eventually replaced by scar tissue.

Within seconds of a myocardial infarction, the under-perfused myocardial cells no longer contract, leading to abnormal wall motion, high wall stresses within and surrounding the infarct, and depressed ventricular function. The high stresses at the junction between the infarcted tissue and the normal tissue lead to expansion of the infarcted area and to remodeling of the heart over time. These high stresses eventually injure the still viable myocardial cells depress their function. This results in an expansion of injury and dysfunctional tissue including and beyond the original myocardial infarct region.

According to the American Heart Association, in the year 2000 approximately 1,100,000 new myocardial infarctions occurred in the United States. For 650,000 patients this was their first myocardial infarction, while for the other 450,000 patients this was a recurrent event. Two hundred-twenty thousand people suffering MI die before reaching the hospital. Within one year of the myocardial infarction, 25% of men and 38% of women die. Within 6 years, 22% of men and 46% of women develop heart failure, of which 67% are disabled. This is despite modern medical therapy.

The consequences of myocardial infarction are often severe and disabling. When a myocardial infarction occurs, the myocardial tissue that is no longer receiving adequate blood flow dies and is replaced with scar tissue. This infarcted tissue cannot contract during systole, and may actually undergo lengthening in systole and leads to an immediate depression in ventricular function. This abnormal motion of the infarcted tissue can cause delayed or abnormal conduction of electrical activity to the still surviving peri-infarct tissue (tissue at the junction between the normal tissue and the infarcted tissue) and also places extra structural stress on the peri-infarct tissue.

Thus, in addition to immediate hemodynamic effects, the infarcted tissue and the myocardium or cardiac tissue undergo three major processes: infarct expansion, infarct extension, and chamber remodeling. These factors individually and in combination contribute to the eventual dysfunction observed in the cardiac tissue remote from the site of the infarction

Infarct expansion is a fixed, permanent, disproportionate regional thinning and dilatation of tissue within the infarct zone. Infarct expansion occurs early after a myocardial infarction. The mechanism is slippage of the tissue layers.

Infarct extension is additional myocardial necrosis following myocardial infarction. Infarct extension results in an increase in total mass of infarcted tissue and the additional infarcted tissue may also undergo infarct expansion. Infarct extension occurs days after a myocardial infarction. The mechanism for infarct extension appears to be an imbalance in the blood supply to the peri-infarct tissue versus the increased oxygen demands on the tissue.

Remodeling is usually the progressive enlargement of the ventricle accompanied by a depression of ventricular function. Myocyte function in the cardiac tissue remote from the initial myocardial infarction becomes depressed. Remodeling occurs weeks to years after myocardial infarction. Such remodeling usually occurs on the left side of the heart. Where remodeling does occur on the right side of the heart, it can generally be linked to remodeling (or some other negative event) on the left side of the heart. Remodeling can occur independently in the right heart, albeit less often than the left. There are many potential mechanisms for remodeling, but it is generally believed that the high stress on peri-infarct tissue plays an important role. Due to variety of factors such as altered geometry, wall stresses are much higher than normal in the cardiac tissue surrounding the infarction.

The processes associated with infarct expansion and remodeling are believed to be the result of high stresses exerted at the junction between the infarcted tissue and the normal cardiac tissue (i.e., the peri-infarct region). In the absence of intervention, these high stresses will eventually kill or severely depress function in the adjacent cells. As a result, the peri-infarct region will therefore grow outwardly from the original infarct site over time. This resulting wave of dysfunctional tissue spreading out from the original myocardial infarct region greatly exacerbates the nature of the disease and can often progress into advanced stages of heart failure.

The treatments for myocardial infarction that are used currently, and that have been used in the past, are varied. Immediately after a myocardial infarction, preventing and treating ventricular fibrillation and stabilizing the hemodynamics are well-established therapies.

Newer approaches include more aggressive efforts to restore patency to occluded vessels. This is accomplished through thrombolytic therapy or angioplasty and stents. Reopening the occluded artery (i.e. revascularization) within hours of initial occlusion can decrease tissue death, and thereby decrease the total magnitude of infarct expansion, extension, and thereby limit the stimulus for remodeling.

Surgical approaches to exclude, to isolate, or to remove the infarct region have been proposed. Other surgical treatments envision surrounding the heart, or a significant portion thereof, with a jacket or mesh type prosthesis to prevent remodeling. Other potential surgical approaches include the application of heat to shrink the infarcted tissue, followed by the suturing of a patch onto the infarcted region. Surgical approaches for accessing the heart vary widely. For example, one method of surgical access to the heart may be through a median sternotomy (open-chest surgical exposure) or a thoracotomy. A median sternotomy incision begins just below the sternal notch and extends slightly below the xyphoid process. A sternal retractor is used to separate the sternal edges for optimal exposure of the heart. Hemostasis of the sternal edges is typically obtained using electrocautery with a ball-tip electrode and a thin layer of bone wax.

The open chest procedure generally involves making a 20 to 25 cm incision in the chest of the patient, severing the sternum and cutting and peeling back various layers of tissue in order to give access to the heart and arterial sources. As a result, these operations typically require large numbers of sutures or staples to close the incision and 5 to 10 wire hooks to keep the severed sternum together. Such surgery often carries additional complications such as instability of the sternum, post-operative bleeding, and mediastinal infection. The thoracic muscle and ribs are also severely traumatized, and the healing process results in an unattractive scar. Post-operatively, most patients endure significant pain and must forego work or strenuous activity for a long recovery period.

Many minimally invasive surgical techniques and devices have been introduced in order to reduce the risk of morbidity, expense, trauma, patient mortality, infection, and other complications associated with open-chest cardiac surgery. Less traumatic limited open chest techniques using an abdominal (sub-xyphoid) approach or, alternatively, a “Chamberlain” incision (an approximately 8 cm incision at the sternocostal junction), have been developed to lessen the operating area and the associated complications. In recent years, a growing number of surgeons have begun performing coronary artery bypass graft (CABG) procedures using minimally invasive direct coronary artery bypass grafting (MIDCAB) surgical techniques and devices. Using the MIDCAB method, the heart typically is accessed through a mini-thoracotomy (i.e., a 6 to 8 cm incision in the patient's chest) that avoids the sternal splitting incision of conventional cardiac surgery. A MIDCAB technique for performing a CABG procedure is described in U.S. Pat. No. 5,875,782, for example.

Other minimally invasive, percutaneous, coronary surgical procedures have been advanced that employ multiple small trans-thoracic incisions to and through the pericardium, instruments advanced through ports inserted in the incisions, and a thoracoscope to view the accessed cardiac site while the procedure is performed as shown, for example, in U.S. Pat. Nos. 6,332,468, 5,464,447, and 5,716,392. Surgical trocars having a diameter of about 3 mm to 15 mm are fitted into lumens of tubular trocar sleeves, cannulae or ports, and the assemblies are inserted into skin incisions. The trocar tip is advanced to puncture the abdomen or chest to reach the pericardium and the trocar is then withdrawn leaving the sleeve or port in place. Surgical instruments and other devices such as fiber optic thoracoscopes can be inserted into the body cavity through the sleeve or port lumens. As stated in the '468 patent, instruments advanced through trocars can include electrosurgical tools, graspers, forceps, scalpels, electrocauteries, clip appliers, scissors, etc.

In such procedures, the surgeon can stop the heart by utilizing a series of internal catheters to stop blood flow through the aorta and to administer cardioplegia solution. The endoscopic approach utilizes groin cannulation to establish cardio-pulmonary bypass (CPB) and an intraaortic balloon catheter that functions as an internal aortic clamp by means of an expandable balloon at its distal end used to occlude blood flow in the ascending aorta. A full description of an example of one preferred endoscopic technique is found in U.S. Pat. No. 5,452,733, for example.

Problems may develop during CPB due to the reaction blood has to non-endothelially lined surfaces, i.e., surfaces unlike those of a blood vessel. In particular, exposure of blood to foreign surfaces results in the activation of virtually all the humoral and cellular components of the inflammatory response, as well as some of the slower reacting specific immune responses. Other complications from CPB include loss of red blood cells and platelets due to shear stress damage. In addition, cardiopulmonary bypass requires the use of an anticoagulant, such as heparin. This may, in turn, increase the risk of hemorrhage. Finally cardiopulmonary bypass sometimes necessitates giving additional blood to the patient. The additional blood, if from a source other than the patient, may expose the patient to bloodborne diseases.

Due to the risks incurred during CPB, some surgeons have attempted to perform cardiac-related surgical procedures without cardiac arrest and CPB. For example, Trapp and Bisarya in “Placement of Coronary Artery Bypass Graft without Pump Oxygenator”, Annals Thorac. Surg. Vol. 19, No. 1, (January 1975) pgs. 1-9, immobilized the area of a bypass graft by encircling sutures deep enough to incorporate enough muscle to suspend an area of the heart and prevent damage to the coronary artery. More recently Fanning et al. in “Reoperative Coronary Artery Bypass Grafting Without Cardiopulmonary Bypass”, Annals Thorac. Surg. Vol. 55, (Feb. 1993) pgs. 486-489 also reported immobilizing the area of a bypass graft with stabilization sutures.

Suction stabilization systems, such as the Medtronic Octopus® Tissue Stabilizer and the Medtronic Starfish® and Urchin® Heart Positioners (available from Medtronic, Inc., Minneapolis, Minn. USA) use suction to grip and immobilize the surface of the heart. Additionally, the system allows the surgeon to manipulate the surgical site into better view by rotating and supporting the heart. See, also, e.g., U.S. Pat. Nos. 5,836,311; 5,927,284, 6,015,378, 6,464,629 and 6,471,644 and co-assigned U.S. patent application Ser. No. 09/678,203, filed Oct. 2, 2000; and European Patent Publication No. EP 0 993 806. The Octopus® stabilizer and Starfish® and Urchin® positioners facilitate moving or repositioning the heart to achieve better access to areas which would otherwise be difficult to access, such as the posterior or backside of the heart.

The recently developed beating heart procedures also disclosed in U.S. Pat. No. 6,394,948, for example, eliminate the need for any form of CPB, the extensive surgical procedures necessary to connect the patient to a CPB machine, and to stop the heart. These beating heart procedures can be performed on a heart exposed in a full or limited thoracotomy or accessed percutaneously.

In some percutaneous procedures, the epicardium of the beating or stopped heart is exposed to view typically by use of grasping and cutting instruments. These instruments are inserted through one port to cut through the pericardium while the area is viewed through a thoracoscope or endoscope inserted through another port. The thoracoscopic approach typically requires the placement of a chest tube and admission to the hospital for the initial 1-2 post-operative days.

The immediate effects of a blocked coronary artery can be addressed through percutaneous transluminal coronary angioplasty (PTCA). PTCA can be used to dilate an occluded coronary artery, often in conjunction with stenting, to provide blood flow to cardiac cells downstream of the blockage. Alternatively, the coronary artery bypass grafting (CABG) procedure may be used to bypass a blocked coronary artery altogether. More intermediate term damage can be addressed through the systemic or local delivery of agents to reduce or treat the cells affected by the initial injury. The longer-term problems, for example, heart failure resulting from remodeling of infarcted cardiac tissue, have been partially addressed by the systemic or local delivery of medical agents to the cardiac tissue. Some treatments include pharmaceuticals such as ACE inhibitors, beta blockers, diuretics, and Ca⁺⁺ channel antagonists. These agents have multiple effects, but share in the ability to reduce aortic pressure, and thereby cause a slight decease in wall stress. These agents have been shown to slow remodeling, at least partially. However, drug compliance is far from optimal.

The direct or selective delivery of agents to cardiac tissue is often preferred over the systemic delivery of such agents for several reasons. One reason is the substantial expense and small amount of the medical agents available, for example, agents used for gene therapy. Another reason is the substantially greater concentration of such agents that can be delivered directly into cardiac tissue, compared with the dilute concentrations possible through systemic delivery. Yet another reason is that systemic administration is associated with systemic toxicity at doses required to achieve desired drug concentrations in the cardiac tissue.

One mode of delivering medical agents to cardiac tissue is by epicardial, direct injection into cardiac tissue during an open chest procedure. As discussed above, open chest procedures are inherently traumatic procedures with significant associated risks. The risks are often justified if the procedure results in a significant enough benefit to the patient, such as that of a life-saving nature.

Another approach taken to deliver medical agents into cardiac tissue has been an intravascular approach. Catheters may be advanced through the vasculature and into the heart to inject materials into cardiac tissue from within the heart. This approach may not allow all areas of the heart to be easily reached however. The size and type of instruments that can be advanced, for example, from a femoral artery approach, are also limited.

Newer therapies for treating infarcted cardiac tissue include the injection of cells and/or other biologic agents into ischemic cardiac tissue or placement of cells and/or agents onto the ischemic tissue. One therapy for treating infarcted cardiac tissue includes the delivery of cells that are capable of maturing into actively contracting cardiac muscle cells. Examples of such cells include myocytes, myoblasts, mesenchymal stem cells, and pluripotent cells. Delivery of such cells into cardiac tissue is believed to be beneficial, particularly to prevent or treat heart failure. Current intravascular delivery devices are less than optimal, being limited in the cardiac regions they can access and the amount and types of materials they can deliver. Open chest procedures allow access to a larger range of cardiac tissue and also allow the delivery of greater varieties and amounts of agents, for example, cells. An open chest procedure may not be justifiable, however, only for the delivery of such cells. In particular, patients having suffered a recent heart attack may be very poor candidates for such a procedure.

Despite improvements in therapy, the incidence and prevalence of heart failure continues to rise with over 400,000 new cases each year. Approximately 85% of these new cases are due to ischemic cardiomyopathy.

At present, there are no available procedures that provide both structural stabilization and biological therapy to injured cardiac tissue to prevent myocardial extension and remodeling. Such treatments would be advantageous over previously used treatments. For this reason, it is desirable to have an agent that could be delivered to cardiac tissue to provide structural stabilization and/or biological therapy of injured cardiac tissue to address myocardial extension and remodeling. In particular, devices and methods enabling a minimally invasive delivery of one or more agents into cardiac tissue would be most advantageous.

### SUMMARY OF THE INVENTION

It is accordingly an object of the present invention to disclose devices and methods for providing structural reinforcement to injured tissue.

It is also an object of the present invention to disclose devices and methods for providing structural reinforcement of tissue alone to injured tissue

It is a further object of the present invention to disclose methods for providing structural reinforcement of tissue in combination with biological therapy to injured tissue

It is a further object of the present invention to disclose methods for treating injured cardiac tissue.

It is a further object of the present invention to disclose methods for treating injured cardiac tissue using percutaneous transluminal delivery devices and techniques.

It is a further object of the present invention to disclose methods for treating injured cardiac tissue using epicardial delivery devices and techniques.

It is a further object of the present invention to disclose methods for treating injured cardiac tissue using endocardial delivery devices and techniques.

Yet another object of the present invention to disclose methods for treating injured cardiac tissue using surgical and minimally invasive surgical techniques.

A further object of the present invention is to provide structural reinforcement of the tissue to injured cardiac tissue and such structural reinforcement of the tissue in combination with biological therapy by using substances containing platelets.

The current invention satisfies those objects by disclosing methods of providing structural reinforcement of the tissue to injured cardiac tissue, and structural reinforcement of the tissue combined with biological therapy to injured cardiac tissue.

The present invention discloses delivering a composition (injectate) into an injured, ischemic (blood flow reduced enough to cause cell death and/or tissue necrosis), or infarcted (blood flow eliminated and necrosis or death of cells and tissue has occurred) heart to provide such structural support and/or therapy. Because the methods of treatment described herein can be used for both ischemic and infarcted myocardium or cardiac tissue, they will be collectively referred to hereinafter as injured myocardium or injured tissue.

In various embodiments of the present invention, the composition can be delivered to the injured region of cardiac tissue, the peri-injury region (i.e., the region of tissue directly adjacent to the ischemic tissue), or healthy tissue. While this document discusses embodiments of the present invention in relation to chamber remodeling, particularly remodeling of the ventricle on the left side of the heart, it is to be understood that the devices and methods of the present invention are applicable to other areas and parts of the heart, including other chambers, valves and structures.

Another aspect of the current invention accomplishes the objects by delivering a composition into cardiac tissue having a region of ischemic tissue for the sole purpose of providing structural reinforcement of the tissue. The composition can comprise or include platelet gel, other substances described herein, or any substance suitable for providing the desired level of support.

In general, platelet gel is formed by activating plasma that contains platelets, e.g., platelet rich plasma (PRP) or platelet poor plasma (PPP), with a clot promoting activator or agent, e.g., thrombin. When blood is collected and spun in a centrifuge to separate the various components such as red blood cells, white blood cells, the plasma and the platelets, one can make both PRP and PPP from those components. When PRP is combined with clotting agents to create a “platelet gel,” it can be used to enhance the healing process of various wounds, e.g., surgical wounds. Platelet rich plasma can be made from a patient's own blood to significantly reduce the risk of adverse reactions or infection. When platelet gel is made using PRP from a patient's own blood, it is called autologous platelet gel (APG).

When the clotting agent is combined with the PRP, a thick gel results. For example, platelet gel may be formed by mixing platelet rich plasma with thrombin. The thrombin initiates the clotting cascade and creates a platelet gel. The addition of thrombin to PRP and PPP is further disclosed in U.S. Pat. No. 6,444,228, the disclosure of which is incorporated herein by reference.

Since it would be difficult to pass a gel through a needle, it is desirable to inject the PRP or PPP and thrombin into tissue before it forms a gel. Additionally, the spreading and gelling of the delivered composition in situ facilitates greater distribution of the composition and thus greater mechanical and/or biological support. Therefore, methods for causing the plasma and platelets to gel within the tissue are desirable. Mixing a clotting agent or activator, e.g., thrombin, with the plasma and platelets immediately prior to administration can be desirable. For example, thrombin and PRP may be mixed at the very tips of two separate injection needles lying adjacent each other wherein thrombin is delivered via one needle and PRP is delivered via the second needle. In this case, the PRP may permeate tissue at the injection site prior to gelling. Alternatively, thrombin may be injected into tissue through one or more needles and the PRP injected through a different needle or needles.

The rationale for using platelet gel in embodiments of the present invention is that platelet gel provides many features and promoters of healthy healing that may be beneficial in preventing ventricular remodeling after a myocardial infarction or other ischemic insult. These include the structural support that the “gel” itself provides. The platelet gel (alone or augmented by additives described herein) provides a biocompatible support to surrounding tissue, that may resist dilatation and permit a more healthy distribution of forces as the injured ventricle adjusts to sub-optimal forces post-myocardial infarction (MI). In one embodiment, the composition is delivered in an amount sufficient to create an internal “cast” which resists excessive dilation and/or myocardial stretch and thus deters one or more of the triggers of remodeling.

Additionally, platelet gel provides many biologically active agents released from the activated platelets which can facilitate healthy healing and potentially local regeneration. These agents include, but are not limited to, cytokines (including IL-1β, IL-6, TNF-α), chemokines (including ENA-78 (CXCL5), IL-8 (CXCL8), MCP-3 (CCL7), MIP-1α(CCL3), NAP-2 (CXCL7), PF4 (CXCL4), RANTES (CCL5)), inflammatory mediators (including PGE2), and growth factors (including Angiopoitin-1, bFGF, EGF, HGF, IGF-I, IGF-II, PDGF AA and BB, TGF-β1, 2, and 3, and VEGF). Any or all of these are normally responsible for facilitating wound healing. Whether local activity of these agents or recruitment of circulating cells to the injured site or stimulation of local angiogenesis results as part of this mechanism, it is very likely that the biological milieu provided by the platelet gel will lead to more healthy remodeling than occurs in its absence. Additionally, if this platelet gel is provided in an autologous manner (e.g. APG), concerns about rejection, incompatibility and infection are reduced.

In one embodiment, the needles of a delivery device may be interlaced so that the thrombin and the plasma and platelets are injected simultaneously in close proximity. While individual examples disclose the use of PRP, it will be understood by persons of ordinary skill in the art that PPP is also suitable for forming the platelet gel according to the teachings of the present invention. For example, a device may be designed to comprise alternating PRP and thrombin needles spaced 2 mm apart such that mixing will occur within the tissue around the injection site. In one embodiment, delivery devices may include two or more syringes for the delivery of two or more components of the composition, such as PRP and thrombin to a treatment site. For example, a first syringe may be used to deliver PRP to a first set of needles while a second syringe may be used to deliver thrombin to a second set of needles. In one embodiment, the first and second sets of needles are interlaced with each other in a needle array. In another embodiment, the first and second sets of needles alternate with each other along a row. In an alternative embodiment, two or more syringes with needles may be used sequentially. For example, PRP may be delivered with a first syringe to a treatment site followed by a second syringe used to deliver thrombin to the same site.

In one embodiment PRP, may be injected into tissue without the addition of any exogenous clotting agent or agents, since there are initiators within the extracellular matrix to cause the plasma to gel. In an alternative embodiment, a clotting protein, e.g. fibrinogen may be injected into tissue. Fibrinogen may be injected into tissue with or without the addition of a clotting (gelling) agent, for example, thrombin. In one embodiment, fibrinogen is delivered in combination with PRP (with or without thrombin).

Another aspect of the current invention achieves the above stated objects by disclosing a predominantly acellular approach to preventing cardiac remodeling by providing platelet gel into an injured, ischemic, or infarcted heart. The platelet gel will provide structural reinforcement of the tissue and it may provide biological therapy to prevent post-injury chamber remodeling.

A further aspect of the present invention provides a method for treating injured tissue by providing a platelet gel comprising PRP and thrombin into the injured tissue. The PRP and thrombin may be delivered separately to a common site in the recipient cardiac tissue for rapid in situ coagulation into platelet gel. In various embodiments of this aspect of the present invention, the PRP can be derived from a source other than the recipient (e.g., recombinant, animal, human, engineered, etc.) or it can be autologous. Additionally, the thrombin can be derived from a source other than the recipient (e.g., recombinant, animal, human, engineered, etc.), or it can be autologous. In one embodiment, bovine thrombin is used.

Another aspect of the present invention provides for delivering a composition to injured cardiac tissue immediately after restoration of blood flow (i.e., after revascularization) or sometime later. In one embodiment, the composition is delivered at the time of revascularization within the same procedure. In other embodiments the composition can be administered hours, days, weeks, months, or years afterwards in a separate procedure following spontaneous or procedural revascularization. One embodiment provides for delivering the composition to cardiac tissue prior to ischemic injury. The intended use is to select timing of administration so as to optimize clinical effect.

A further aspect of the present invention provides a device for delivering a composition having multiple components that cannot be mixed until just prior to or after being injected, such a composition may be delivered using a multi-lumen injector to combine the components at the site of tissue delivery. An example of such a composition would be platelet gel made from PRP and thrombin. In one embodiment, a 10:1 ratio of PRP to thrombin is employed, and other embodiments can use different ratios of PRP to thrombin. Additional embodiments of the present invention may include other materials in the injected substance either as substitutes for, or in addition to, the PRP and thrombin.

Other preferred embodiments of the present invention provide for mixing the constituent components, of a multi-component composition, immediately prior to delivering the composition to the cardiac tissue.

Still further aspects of the present invention provide for delivery of the composition using minimally invasive surgical techniques, e.g., endoscopic, thoracoscopic, port access, small incision and/or sub-xiphoid approaches or using percutaneous transluminal delivery devices, transvascular delivery devices, endocardial devices, and/or epicardial devices.

Additional aspects of the present invention provide for delivery of the composition using one or more injections. A single injection may be sufficient or multiple injections may be necessary to deliver an appropriate dose in divided injections.

One embodiment of the present invention targets delivering a fixed dose (volume) of composition per patient, allowing the operator to adjust the volume and number of injection sites to deliver that target dose. Another embodiment of the present invention targets delivering a fixed injection volume per site. Another embodiment of the present invention targets delivering a fixed number of injection sites or number of sites per injured tissue volume. One embodiment of the present invention targets mid-wall thickness injections (i.e., if the wall is 1 cm thick, 5 mm needle penetration).

In at least one embodiment of the present invention, the composition is injected only into injured cardiac tissue, while in other embodiments the peri-injury zone around an injured region is injected, and in at least one embodiment of the present invention, the composition is injected into only the healthy tissue. In other embodiments, the composition can be delivered to any combination of the regions of ischemic cardiac tissue, tissue in the peri-infarct zone, and healthy tissue.

In one embodiment of the present invention, a method is provided for preventing chamber remodeling of the heart due to myocardial injury by structurally reinforcing the cardiac tissue comprising providing at least one composition into a treatment site in the cardiac tissue wherein said composition provides structural support to the cardiac tissue.

In an embodiment of the present invention, the composition comprises one or more than one structural material selected from the group consisting of platelet gel, autologous platelet gel, collagen, biocompatible polymers, alginates, synthetic/natural compounds, fibrinogen, silk-elastin polymers, hydrogels and dental composite materials.

In another embodiment of the present invention, the composition is injected at the treatment site and forms a solid or a gel within the cardiac tissue at the treatment site. In another embodiment, the composition forms a solid or a gel as a result of physical or chemical cross-linking or enzymatic, chemical, thermal or light activation of the composition.

In one embodiment of the present invention, the composition comprises autologous platelet gel. In another embodiment, the autologous platelet gel is formed from platelet poor plasma or platelet rich plasma and an activating agent. In another embodiment, the platelet rich plasma or said platelet poor plasma is freshly isolated and the activating agent is thrombin. In one embodiment the thrombin is selected from the group consisting of recombinant thrombin, extracted animal thrombin, engineered thrombin and autologous thrombin.

In another embodiment, the at least one composition comprises two or more compositions and the two or more compositions are injected approximately simultaneously at the treatment site.

In an embodiment of the present invention, the composition further comprises a bioactive agent. In another embodiment, the bioactive agent is selected from the group consisting of pharmaceutically active compounds, hormones, growth factors, enzymes, DNA, RNA, siRNA, viruses, proteins, lipids, polymers, hyaluronic acid, antibodies, antibiotics, anti-inflammatory agents, anti-sense nucleotides and transforming nucleic acids, inhibitors of compounds implicated in remodeling (e.g. angiotensin II, angiotensin converting enzyme, atrial natriuretic peptide, aldosterone, renin, norepinephrine, epinephrine, endothelin, etc.), and combinations thereof.

In another embodiment of the present invention, the composition further comprises a contrast agent. In another embodiment, the composition further comprises an agent to increase the structural strength of the composition. In another embodiment the agent to increase the structural strength of the composition is fibrinogen.

In another embodiment of the present invention, the composition is provided to the injured cardiac tissue between 1 hour and 2 weeks after the damage to the heart occurs. In another embodiment, the composition is provided in 1 to 20 injections. In another embodiment, the injections are provided sequentially. In yet another embodiment, the injections are provided approximately simultaneously. In another embodiment, the composition comprises a total injection volume up to 15 mL. In another embodiment, the composition comprises an injection volume up to 1100 microliters per injection. In another embodiment, the composition is injected into the cardiac tissue at an angle orthogonal or oblique to the cardiac surface.

In another embodiment of the present invention, the composition is provided to a treatment site in the cardiac tissue selected from the group consisting of sub-endocardial, sub-epicardial and intra-myocardial sites. In yet another embodiment, the composition is injected into the cardiac tissue at a depth midway through the thickness of the myocardium. In another embodiment, the composition is injected into the cardiac tissue at a depth other than midway through the thickness of the myocardium.

In yet another embodiment of the present invention, the ratio of platelet rich plasma or platelet poor plasma to thrombin is between approximately 5:1 to approximately 25:1. In another embodiment, the ratio of platelet rich plasma or platelet poor plasma to thrombin is between approximately 10:1.

In another embodiment of the present invention, the method further comprises a delivery device adapted to deliver the composition into the injured cardiac tissue. In another embodiment, the delivery device is an injection catheter selected from the group consisting of an endocardial injection catheter, a transvascular injection catheter and an epicardial injection catheter.

In yet another embodiment of the present invention, the composition is provided to the treatment site during an injurious event or after an injurious event has occurred. In another embodiment, treatment site is selected from the group consisting of the injured area, the peri-injury area and the healthy tissue surrounding the injured area.

In one embodiment of the present invention, a system is provided for preventing chamber remodeling of the heart due to cardiac tissue injury by structurally reinforcing the cardiac tissue comprising at least one composition and at least one delivery device for introducing the composition into the cardiac tissue and wherein the composition provides structural support for the cardiac tissue.

In another embodiment of the present invention, the system further comprises a cardiac stabilization device. In another embodiment, the system further comprises an imaging device. In another embodiment, the imaging device is an echocardiography device.

In an embodiment of the present invention, the composition comprises one or more than one structural material selected from the group consisting of platelet gel, autologous platelet gel, collagen, biocompatible polymers, alginates, synthetic/natural compounds, fibrinogen, silk-elastin polymers, hydrogels and dental composite materials.

In another embodiment of the present invention, the composition is injected at the treatment site and forms a solid or a gel within the cardiac tissue at the treatment site. In another embodiment, the composition forms a solid or a gel as a result of physical or chemical cross-linking or enzymatic, chemical, thermal or light activation of the composition.

In one embodiment of the present invention, the composition comprises autologous platelet gel. In another embodiment, the autologous platelet gel is formed from platelet poor plasma or platelet rich plasma and an activating agent. In another embodiment, the platelet rich plasma or said platelet poor plasma is freshly isolated and the activating agent is thrombin. In one embodiment the thrombin is selected from the group consisting of recombinant thrombin, extracted animal thrombin, engineered thrombin and autologous thrombin.

In another embodiment, the at least one composition comprises two or more compositions and the two or more compositions are injected approximately simultaneously at the treatment site.

In an embodiment of the present invention, the composition further comprises a bioactive agent. In another embodiment, the bioactive agent is selected from the group consisting of pharmaceutically active compounds, hormones, growth factors, enzymes, DNA, RNA, siRNA, viruses, proteins, lipids, polymers, hyaluronic acid, antibodies, antibiotics, anti-inflammatory agents, anti-sense nucleotides and transforming nucleic acids, inhibitors of compounds implicated in remodeling (e.g. angiotensin II, angiotensin converting enzyme, atrial natriuretic peptide, aldosterone, renin, norepinephrine, epinephrine, endothelin, etc.), and combinations thereof.

In another embodiment of the present invention, the composition further comprises a contrast agent. In another embodiment, the composition further comprises an agent to increase the structural strength of the composition. In another embodiment the agent to increase the structural strength of the composition is fibrinogen.

In another embodiment of the present invention, the composition is provided in 1 to 20 injections. In another embodiment, the injections are provided sequentially. In yet another embodiment, the injections are provided approximately simultaneously. In another embodiment, the composition comprises a total injection volume up to 15 mL. In another embodiment, the composition comprises an injection volume up to 1100 microliters per injection. In another embodiment, the composition is injected into the cardiac tissue at an angle orthogonal or oblique to the heart surface.

In another embodiment of the present invention, the composition is provided to a treatment site in the cardiac tissue selected from the group consisting of sub-endocardial, sub-epicardial and intra-myocardial sites. In yet another embodiment, the composition is injected into the cardiac tissue at a depth midway through the thickness of the myocardium. In another embodiment, the composition is injected into the cardiac tissue at a depth other than midway through the thickness of the myocardium.

In yet another embodiment of the present invention, the ratio of platelet rich plasma or platelet poor plasma to thrombin is between approximately 5:1 to approximately 25:1. In another embodiment, the ratio of platelet rich plasma or platelet poor plasma to thrombin is between approximately 10:1. The injected substances of the current invention can be made from autologous, non-autologous, or recombinant substances. One advantage in using autologous and/or recombinant components in the injected substances is that it reduces a recipient's risk of exposure to communicable disease.

In another embodiment of the present invention, the system further comprises a delivery device adapted to deliver the composition into the injured cardiac tissue. In another embodiment, the delivery device is an injection catheter selected from the group consisting of an endocardial injection catheter, a trans-vascular injection catheter, and an epicardial injection catheter.

In yet another embodiment of the present invention, the composition is provided to the treatment site during an injurious event or after an injurious event has occurred. In another embodiment, treatment site is selected from the group consisting of the injured area, the peri-injury area and the healthy tissue surrounding the injured area.

The present invention discloses methods and devices for treating injured cardiac tissue by injecting the cardiac tissue with a composition to provide structural reinforcement of the tissue or structural reinforcement of the tissue combined with biological therapy. The foregoing and other features and advantages of the present invention will become further apparent from the following detailed description of the presently preferred embodiments, read in conjunction with the accompanying drawings, which are not to scale. The detailed description and drawings are merely illustrative of the invention, rather than limiting the scope of the invention being defined by the appended claims and equivalents thereof.

### BRIEF DESCRIPTION OF THE DRAWINGS

FIG. 1 is a drawing of a normal, healthy heart.

FIG. 2 is a drawing of a heart with a region of injured myocardium.

FIG. 3 is an enlarged view of the injured myocardium depicted in FIG. 2.

FIG. 4 is a cross-sectional depiction of the heart shown in FIG. 1.

FIG. 5 is a cross-sectional depiction of a heart showing a region of injured and remodeled cardiac tissue on the wall of the left ventricle. Eligible injured cardiac tissue can be of different thicknesses and geometries. One example is shown with a mildly thinned and dilated (aneurismal) wall.

FIG. 6 depicts a needle being used to deliver a composition to an injured myocardium according to an embodiment of the current invention

FIG. 7 depicts a needle being used to deliver a composition to an injured myocardium according to an embodiment of the current invention.

FIG. 8 shows a device used for stabilizing a target cardiac tissue, relative to an injection needle, during delivery of a composition according to the teachings of the present invention.

FIG. 9 depicts a positioning device positioning a heart into a non-physiological orientation.

FIG. 10 is a block diagram showing the steps of treating an injured cardiac tissue according to the teachings of the current invention.

FIG. 11 schematically depicts delivery of a composition into an injured heart according to one embodiment of the present invention.

FIG. 12 schematically depicts a detailed view of delivery of a composition into injured cardiac tissue according to another embodiment of the present invention.

FIG. 13 schematically depicts the migration of a composition within the myocardial tissue after delivery into injured cardiac tissue according to an embodiment of the present invention.

FIG. 14 schematically depicts an epicardial approach to delivery of compositions to injured cardiac tissue according to the teachings of the present invention.

FIG. 15A-B schematically depicts an endocardial approach to delivery of compositions to injured cardiac tissue according to the teachings of the present invention. FIG. 15A depicts an anterograde endocardial approach through the venous system and FIG. 15B depicts a retrograde endocardial approach through the arterial system.

FIG. 16A-B schematically depicts a transvascular approach to delivery of compositions to injured cardiac tissue according to the teachings of the present invention. FIG. 16A depicts a venous approach and FIG. 16B depicts an arterial approach through the coronary artery.

FIG. 17 depicts a flow diagram of the system of the present invention.

FIG. 18 depicts a photomicrograph of infarcted myocardium eight weeks after injection with autologous platelet gel (platelet rich plasma and bovine thrombin at 10:1 ratio) one hour after infarction according to the teachings of the present invention.

### DEFINITION OF TERMS

Generally, all technical terms or phrases appearing herein are used as one skilled in the art would understand to be their ordinary meaning. For the convenience of the reader, however, selected terms are more specifically defined as follows.

Bioactive agent: As used herein, “bioactive agent” includes therapeutic agents and drugs and includes pharmaceutically active compounds, hormones, growth factors, enzymes, DNA, RNA, siRNA, viruses, proteins, lipids, polymers, hyaluronic acid, antibodies, antibiotics, anti-inflammatory agents, anti-sense nucleotides and transforming nucleic acids, inhibitors of compounds implicated in remodeling (e.g., angiotensin II, angiotensin converting enzyme, atrial natriuretic peptide, aldosterone, renin, norepinephrine, epinephrine, endothelin, etc.) and combinations thereof.

Chamber remodeling: As used herein, “chamber remodeling” refers to remodeling of the atria or ventricles. “Remodeling” refers to a series of events (which may include changes in gene expression, molecular, cellular and interstitial changes) that result in changes in size, shape and function of cardiac tissue following stress or injury. Remodeling may occur after myocardial infarction (MI), pressure overload (e.g., aortic stenosis, hypertension), volume overload (e.g., valvular regurgitation), inflammatory heart disease (e.g., myocarditis), or in idiopathic cases (e.g., idiopathic dilated cardiomyopathy). Remodeling is often pathologic, resulting in progressively worsening cardiac function and ultimately a failing heart. Pathologic remodeling as described above will be referred to as remodeling in this disclosure.

Cardiac tissue injury: As used herein, “cardiac tissue injury” refers to any area of abnormal tissue in the heart caused by a disease, disorder or injury and includes damage to the epicardium, endocardium, and/or myocardium. Non-limiting examples of causes of cardiac tissue injury include acute or chronic stress (systemic hypertension, pulmonary hypertension, valve dysfunction, etc.) coronary artery disease, ischemia or infarction, inflammatory disease and cardiomyopathies. Cardiac tissue injury most often involves injury to the myocardium and therefore, for the purposes of this disclosure, myocardial injury is equivalent to cardiac tissue injury. Furthermore, there are occasions when the injury is acute, such as in an acute myocardial infarction or a myocardial infarction, where the injury may be referred to as an injurious event.

Composition: As used herein, “composition” refers to an injectate, substance or a combination of substances which can be delivered into a tissue and are used interchangeably herein.

Delivery: As used here, “delivery” refers to providing a composition to a treatment site in an injured tissues through any method appropriate to deliver the functional composition to the treatment site. Non-limiting examples of delivery methods include, direct injection at the treatment site, direct topical application at the treatment site, percutaneous delivery for injection, percutaneous delivery for topical application, and other delivery methods well known to persons of ordinary skill in the art.

Injury area: As used herein, “injury area” refers to the injured tissue. The “peri-injury area” refers to the tissue immediately adjacent to the injured tissue. That is, the tissue at the junction between the injured tissue and the normal tissue.

Percutaneous: As used herein, the term “percutaneous” refers to any penetration through the skin of the patient, whether in the form of a small cut, incision, hole, cannula, tubular access sleeve or port or the like. A percutaneous penetration may be made in an interstitial space between the ribs of the patient or it may be made elsewhere, such as the groin area of a patient.

Structural support: As used here, the term “structural support” refers to mechanical reinforcement providing resistance against the stresses and maladaptive processes of remodeling.

### DETAILED DESCRIPTION

The present invention provides biocompatible compositions for treating injured myocardial tissue which provide a structural support for the injured tissue and prevent subsequent loss of cardiac function due to the injury. Associated methods and systems for injecting the biocompatible compositions are also provided.

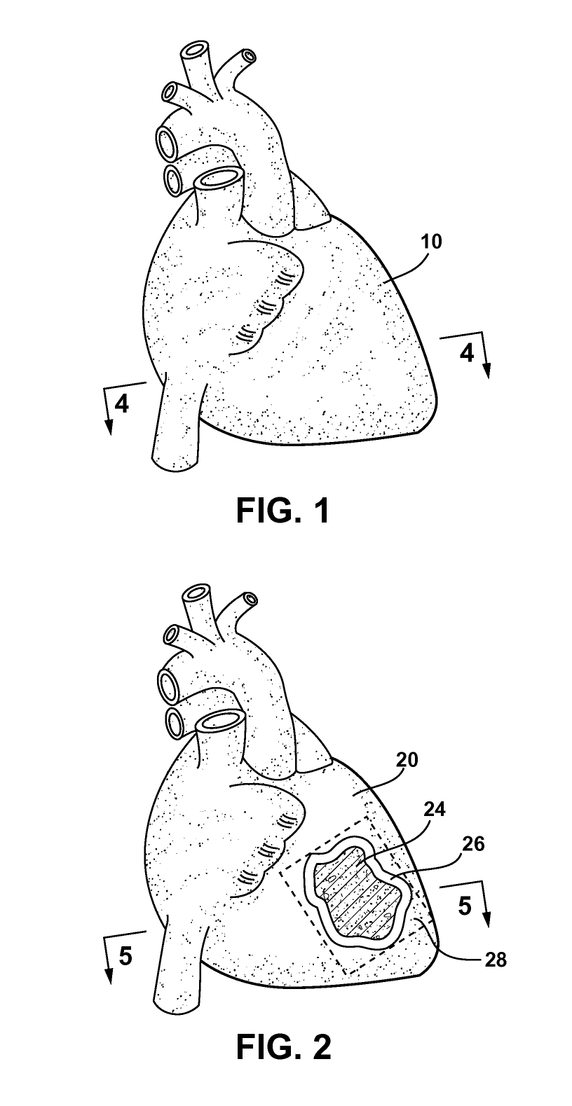

The present invention will now be described in detail below by reference to the drawings, wherein like numbers refer to like structures. Referring to FIGS. 1 and 4, there can be seen depictions of a normal heart 10. The cross-sectional view in FIG. 4 shows the right ventricle 44 and the left ventricle 42 of a normal heart that has not undergone chamber remodeling.

FIG. 2 depicts a heart 20 having an ischemic or infarcted region 24, and a peri-infarct region 26 that is surrounded by healthy non-ischemic myocardium 28. After a myocardial infarction, the ischemic tissue undergoes chamber remodeling as described above. When an infarction occurs, the myocardial tissue that is no longer receiving adequate blood flow dies and is replaced with scar tissue. Triggered by injury, a cascade of events (remodeling) cause the walls to thin, dilate, and ultimately fail.

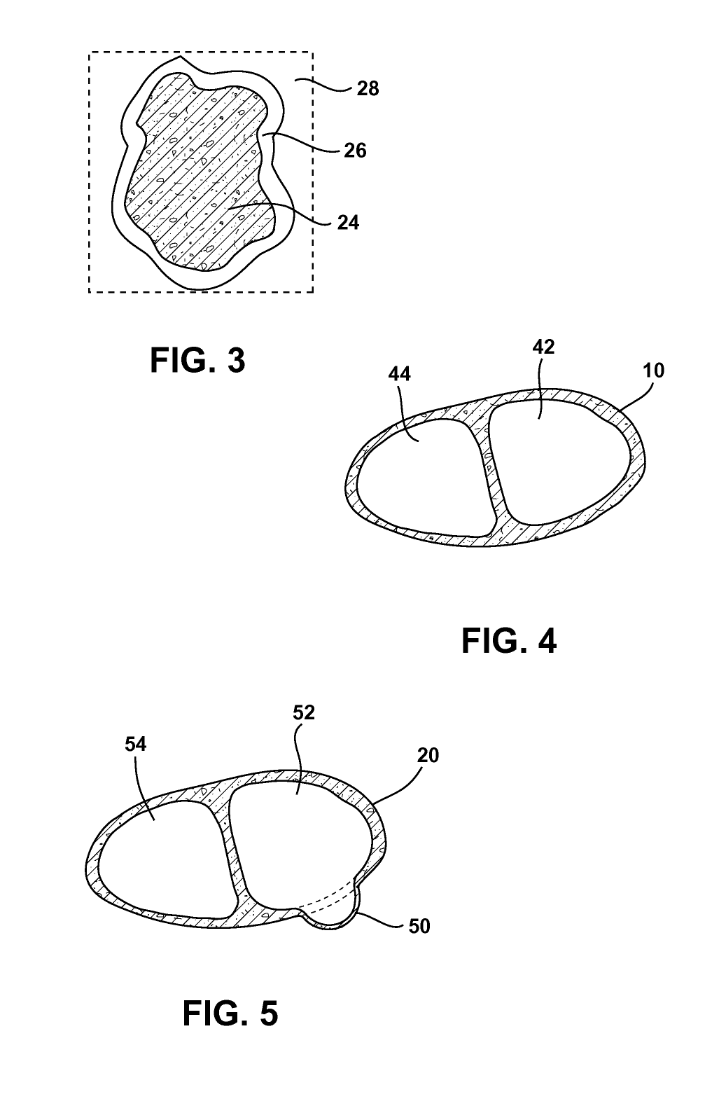

FIG. 3 is an enlarged view of the area bordered by dotted lines in FIG. 2. FIGS. 2 and 3 depict an area of myocardium 24 that has undergone some kind of ischemic insult such as an MI or other injury. If necrosis has occurred, that portion of myocardium that has experienced necrosis will be totally infarcted. The area immediately surrounding the ischemic/infarcted area 26 is known as the peri-infarct area and is surrounded by healthy myocardium 28. As discussed above, the peri-infarct area 26 may have experienced some level of ischemic activity but the blood supply has not yet been interrupted to the same extent as that of the ischemic/infarcted area 24.

Remodeling is usually the progressive enlargement of the ventricle accompanied by a deterioration in ventricular function, and it can occur weeks to years after myocardial infarction. There are many potential mechanisms for remodeling, but it is generally believed that the high stress on peri-infarct tissue plays an important role. Due to altered geometry, wall stresses are much higher than normal in the myocardial tissue surrounding the infarction. FIG. 5 is a cross sectional view of the heart shown in FIG. 2. FIG. 5 shows a right ventricle 54 and a left ventricle 52 having an area 50 of a left ventricle that has undergone remodeling. As can be seen in the figure, the heart walls are thinner in the expanded area 50.

Remodeling is a series of events (which may include gene expression, molecular, cellular, and/or interstitial changes) that result in changes in size, shape, and function of cardiac tissue following stress or injury. Remodeling may occur after myocardial infarction (MI), pressure overload (e.g., aortic stenosis, hypertension), volume overload (e.g., valvular regurgitation), inflammatory heart muscle disease (e.g., myocarditis), or in idiopathic cases (e.g., idiopathic dilated cardiomyopathy). Remodeling is most often “pathologic”, resulting in progressively worsening cardiac function and ultimately a failing heart.

A limited amount of remodeling can be beneficial for the patient and occurs mainly in two contexts: The first is termed “physiologic remodeling” which occurs in some high-performance athletes as an adaptive response to above-normal demands on the heart. The compensatory changes in cardiac geometry and function in the physiologically remodeled heart render it better able to perform in a high-performance environment. The second context is during the earliest stages of post-injury remodeling. Sometimes, the initial phase of this remodeling can actually be adaptive and protective. If to a limited degree, some cellular rearrangement within the cardiac wall and increased chamber volume, can preserve or even augment cardiac output. These changes can be beneficial. However, most often, this progresses beyond what is adaptive to “pathologic remodeling”, in which further changes in wall composition and geometry result in a progressively dysfunctional chamber and eventually a failing heart. “Pathological remodeling” as described above will be referred to in this disclosure as “remodeling.”

Changes associated with remodeling include cardiomyocyte lengthening, cardiac wall thinning, infarct expansion, inflammation and reabsorption of necrotic tissue, scar formation, dilation and reshaping of the chamber (from elliptical towards spherical geometry), myocyte hypertrophy, ongoing myocyte death, and excessive accumulation of collagen. With progressive dilation of, for example, the left ventricle, the end-systolic volume index progressively increases, and the ejection fraction declines. Both of these parameters are important predictors of mortality in humans.

Although the details remain under investigation, the mechanism of remodeling appears to involve a cascade of events. As myocytes stretch under mechanical stress, local activity of several molecules increases (e.g. norepinephrine, angiotensin, endothelin, and others). These molecules stimulate expression of specific proteins and lead to the hypertrophy of existing myocytes. This causes further deterioration in cardiac function (e.g. added mechanical stress) and increased neurohormonal activation. Some of the released factors further stimulate local collagen synthesis which leads to fibrosis and scarring of the affected area. These changes are often beyond compensatory, and lead to a progressively failing heart.

Progressive deterioration in heart function can occur initially in the absence of symptoms. Eventually, however, symptoms of clinical heart failure develop, such as shortness of breath, swelling, difficulty breathing in the supine position, arrhythmias, organ failure, etc. It is important to note that even patients with asymptomatic cardiac dysfunction and milder forms of heart failure are at increased risk of sudden cardiac death. Thus, there is great incentive to treat this disease process early and effectively.

Measures to assess cardiac remodeling include cardiac size, cardiac shape, cardiac mass, ejection fraction, end-diastolic and end-systolic volumes, and peak force of contraction. Left ventricular volume (especially left ventricular end systolic volume) is the best predictor of mortality in humans after myocardial infarction.

Pharmaceutical therapies, which provide angiotensin-converting-enzyme inhibition (e.g., captopril, enalapril) and beta-adrenergic blockade (e.g., carvedilol, metoprolol, propranolol, timolol) have been shown to slow certain parameters of cardiac remodeling. These therapies are intended to reduce the body's remodeling response to injurious or mechanically stressful stimuli and have been shown in clinical trials to reduce mortality and morbidity in myocardial infarction and heart failure patients. Other therapies, such as anti-hypertensive agents, have been used to reduce chronic loads placed on the heart which can trigger or worsen pathologic remodeling. Despite the use of the aforementioned drugs, remodeling remains at best, a process that is partially treatable.

Mechanical methods, such as those described in the present invention, are not routinely employed to prevent or reverse cardiac remodeling in patients. The C or Cap device (manufactured by Acorn Cardiovascular, Inc.), described in U.S. Pat. No. 6,077,218, is an implantable technology comprising a mesh-like device that is placed surgically to fit over the whole heart, providing restraint to the heart globally. However, no existing therapies target specific regions of at-risk or injured tissue, and provide localized mechanical reinforcement to protect against the early steps of the remodeling cascade. Furthermore, no such technologies are available by minimally invasive approaches.

As described further below, embodiments of the present invention address chamber remodeling by injecting a composition into the myocardium to structurally reinforce the tissue by preventing the heart wall from thinning, or by thickening the heart wall, and thus prevent remodeling. The injected composition may occupy some of the interstitial space between the cells of an area of the myocardium and provide structural reinforcement of the tissue. The present invention contemplates providing structural support to any cardiac wall site and includes both the atria and ventricles.

The injected composition can include substances described herein, or any substance suitable for providing the desired structural reinforcement of the tissue. The composition may be a substance that can provide some level of biological therapy as well as the desired structural reinforcement of the tissue. One such substance that may provide both structural reinforcement of the tissue and biological therapy is platelet gel. For the purpose of this document, where the term “platelet gel” is used to describe the present invention in terms of providing structural reinforcement of the tissue or structural reinforcement of the tissue with some biological therapy, the term is used in that context only for the purpose of describing an embodiment of the invention, and it shall be understood to be interchangeable with other substances that have the same or similar properties.

Other compositions suitable for providing the desired structural support can include collagen, cyanoacrylate, adhesives that cure with injection into tissue, liquids that solidify or gel after injection into tissue, suture material, agar, gelatin, light-activated dental composite, other dental composites, silk-elastin polymers, Matrigel® (BD Biosciences), hydrogels and other suitable biopolymers. Such compositions can include single or multi-component compounds. These compositions can include agents that are delivered as a liquid and then gel or harden to a solid after delivery. The hardening/gelling can be triggered by temperature, pH, proteins, or other environmental factors of the tissue where the substance is injected. These compositions can be injected separately or in combination with each other and/or platelet gel. Additionally the compositions or combinations thereof can include other additives. Some of these compositions and/or additives are further described below.

Before any composition is injected into a heart having a region of injured tissue, to provide structural reinforcement of the tissue to the myocardium, the location and extent of the injured region is identified. Multiple technologies and approaches are available for the clinician to identify and assess normal, injured-non-viable, and injured-viable myocardial tissue. These include, but are not limited to, visual inspection during open chest surgical procedures, localized blood flow determinations, local electrical and structural activity, nuclear cardiology, echocardiography, echocardiographic stress test, coronary angiography, magnetic resonance imaging (MRI), computerized tomography (CT) scans, and ventriculography.

In one embodiment of the invention, the platelet gel is prepared by using the Medtronic Magellan® Platelet Separator. Anticoagulated whole blood is prepared by combining an anticoagulant with whole blood freshly removed from the subject. The Magellan® device is used to then extract PRP from the sample of anticoagulated whole blood. Platelet gel is prepared by combining the resulting PRP in an approximately 10:1 ratio with bovine thrombin which has been reconstituted to 1000 Units/milliliter in 10% calcium chloride solution.

Once the location, size and shape of the injured region are identified, the clinician can access and begin injecting the myocardium. If platelet gel is used, it can be comprised of multiple components. In one embodiment, the gel is made using PRP and thrombin alone. The components of the platelet gel may be derived from humans, and/or animals, and/or recombinant sources. The components may also be artificially produced. The components for platelet gel can be categorized as autologous, or non-autologous, and the non-autologous components can be further categorized as described above (i.e., animal, recombinant, engineered, allogeneic human, etc.). One advantage in using autologous and/or recombinant components in the injected compositions is that it reduces the recipient's risk of an inflammatory response or exposure to infectious and foreign agents.

The PRP contains a high concentration of platelets that can aggregate during gelling, as well as release cytokines, growth factors or enzymes following activation. Some of the many factors released by the platelets and the white blood cells that constitute the PRP include platelet-derived growth factor (PDGF), platelet-derived epidermal growth factor (PDEGF), fibroblast growth factor (FGF), transforming growth factor-beta (TGF-β) and platelet-derived angiogenesis growth factor (PDAF). These factors have been implicated in wound healing by increasing the rate of collagen secretion, vascular in-growth and fibroblast proliferation.

The compositions of various embodiments of the current invention can include additives, such as fibrinogen, to increase the structural strength of the myocardium. The fibrinogen can be autologous, allogeneic, recombinant, human, engineered, or purified from animal sources. At least one embodiment includes elastin to increase the elasticity of the treated cardiac wall.

Additionally, the composition can include one or more bioactive agents to induce healing or regeneration of damaged myocardium. Suitable bioactive agents include, but are not limited to, pharmaceutically active compounds, hormones, growth factors, enzymes, DNA, RNA, siRNA, viruses, proteins, lipids, polymers, hyaluronic acid, pro-inflammatory molecules, antibodies, antibiotics, anti-inflammatory agents, anti-sense nucleotides and transforming nucleic acids or combinations thereof. The composition may also include cellular additives such as stem cells, leukocytes, red blood cells, cultured cardiac cells, or other differentiated or undifferentiated cells.

The compositions of the current invention can be fortified with or comprised wholly of a biocompatible liquid that solidifies and/or cross-links in situ to render a structurally supportive structure on delivery into the myocardium. Other embodiments of the composition of the current invention may include synthetic or naturally-occurring materials and/or non-degradable or biodegradable materials to provide strength, for example. In one embodiment, the structural material includes cyanoacrylate or silk-elastin protein polymers.

Furthermore, the compositions of the present invention can include a contrast agent for detection by X-rays, magnetic resonance imaging (MRI) or ultrasound. Suitable contrast agents are known to persons of ordinary skill in the art and include, but are not limited to, radiopaque agents, echogenic agents and paramagnetic agents. A contrast agent may be used in the composition of some embodiments for visual confirmation of injection success. Examples of such contrast agents include, but are not limited to, X-ray contrast (e.g., IsoVue or other contrast agents having a high X-ray attenuation coefficient), MRI contrast (e.g., gadolinium or other contrast agents detectable as signal or signal-void by MRI), and ultrasound contrast (echogenic or echo-opaque compounds

The present invention may be practiced using substances containing synthetic biodegradable materials that provide strength for a specified time interval after delivery, and then resorb. Such materials include genetically-engineered or modified compounds such as collagen or fibrin. Naturally-occurring materials, such as, but not limited to, cartilage, bone or bone components, gelatin, collagen, glycosaminoglycans, starches, polysaccharides, or any other material that provide strength for a specified time interval after delivery, and then resorbs, may also be used.

Other embodiments of the present invention may include a combination of any of a variety of compounds that can create the desired local effect of tissue bulking. Components that cause local edema, thickening of the tissue, structural reinforcement of the tissue, or any other effect that prevents remodeling are included in this invention. Such compounds include ground-up suture material to create edema, hydrogels for structural reinforcement of the tissue. These materials may be added to PRP or PRP+thrombin, or may be used in place of PRP or PRP+thrombin.

If a desired effect is structural reinforcement of the tissue, biodegradable micro-particles between 50-100 μm in size (at the widest point of the particle), such that they are small enough for needle injection but too large to fit into capillaries and venules, may be added to the platelet gel. The micro-particles may be impregnated with a drug that elutes as the particles degrade. In one embodiment of the present invention, micro-particles alone are delivered to the cardiac tissue by injection into the coronary sinus. Based on their size characteristics, they are expected to lodge in the tissue and provide structural reinforcement of the tissue. The micro-particles used may have a glass transition temperature (Tg)≧37° C., so they would gel over days after insertion. The injected micro-particles would provide “mass” and volume for immediate structural reinforcement of the tissue, but soften to gel to become a single member over time.

Embodiments of the composition of the present invention may include polymers that can covalently bind directly to one or more proteins located on the surface of one or more cell types so as to retain the polymers at the local site of injection. In one embodiment, polymers that can covalently bind to the primary amine groups (—NH₃) of proteins may be used.

In one embodiment of the present invention, the composition is a platelet gel that is made using a PRP to thrombin ratio of about 10:1. Another embodiment uses a PRP to thrombin ratio of about 11:1. Other embodiments of the present invention have ratios of PRP to thrombin of about 5:1 to about 25:1. In another embodiment, the ratio of PRP to thrombin is about 7:1 to about 20:1. In another embodiment, the ratio of PRP to thrombin is about 9:1 to about 15:1. In another embodiment, the ration of PRP to thrombin is about 10:1 to about 12:1. In at least one embodiment, no thrombin is included and PRP is injected into the myocardium alone. Other embodiments of the present invention include multiple components in the composition in ratios needed to achieve or optimize the desired effect.

When the PRP and thrombin are injected such that they mix to form platelet gel in the myocardium (see description of delivery devices below) they will gel in the myocardium. Several embodiments of the present invention provide accelerated gel times. The gelling time in situ can be accelerated by applying local heat to the injection site via a delivery catheter or other instrument, increasing the thrombin concentration, or combining the PRP and thrombin in a mixing chamber and injecting the mixture into the myocardium after the mixture has begun gelling. This description also applies for other multi-component compositions, where the components gel, cross-link and/or polymerize after being mixed together.

As described in further detail in the Examples, the compositions of the present invention have been injected into the injured myocardium of test subjects (sheep and pigs). The experiments indicate that injections of PRP and thrombin are safe and well tolerated when made into infarcted or non-infarcted tissue, and that they can be performed safely as early as 1 hr post-MI. Controlled injections were possible with or without a cardiac stabilization device, and it was possible to make the injections without exogenous cardiac pacing. Injections were made both orthogonally and obliquely to the myocardial surface at intervals of 0.5 to 2.5 cm. A plurality of injections can be made per heart without safety problems. The total injectate volume can be as high as 15.0 mL, and the volume of individual injections can be as high as 1100 μl per injection site.

Furthermore, APG administration following myocardial ischemia partially or fully reverses detrimental acute effects of infarction on the ejection fraction (EF), and can augment EF towards or above pre-infarct levels. In a surprising result, APG administration following myocardial injury into ischemic tissue, neovascularization of the ischemic tissue was stimulated (FIG. 18). This neovascularization is not usually observed in infarcted animals not receiving APG therapy.

In order to practice the present invention and deliver a composition to target sites within the myocardium, a clinician may use one of a variety of access techniques. These include surgical (sternotomy, thoracotomy, mini-thoracotomy, sub-xiphoid) approaches and percutaneous (transvascular and endocardial) approaches. Once access has been obtained, the composition may be delivered via epicardial, endocardial, or transvascular approaches. The composition may be delivered to the cardiac wall tissue in one or more locations. This includes intra-myocardial, sub-endocardial, and/or sub-epicardial administration.

In one embodiment of the present invention, a mini-thoracotomy method may be used to deliver the composition. The mini-thoracotomy method includes intubating a patient with a double-lumen endobronchial tube that allows selective ventilation or deflation of the right and left lungs. The left lung is then deflated, thereby helping to provide access to the surface of the heart. A left anterior thoracotomy or incision is created over an intercostal space, preferably the 4th (fourth) intercostal space. An alternative intercostal space may be used depending on the patient's physiology, e.g., the 5th (fifth) intercostal space. The thoracotomy should be as anterior and medial as possible without removing cartilage. A two-inch incision is preferable; however the size of the incision may vary depending on the patient. The ribs, adjacent the incision, may be spread, preferably two-inches or less, using a small rib retractor or spreader to allow adequate access into the chest. If desired, a retractor may be used to spread the ribs both horizontally and vertically. Next, the pericardium is opened directly under the incision. Dissection through fat may be required to reach the pericardium.

The pericardium may be opened by a number of different techniques. In one embodiment of the present invention, the pericardium may be opened by tenting it with graspers and then cutting it with scissors. In an alternative embodiment of the present invention, a device as disclosed in either U.S. Pat. No. 5,931,810 or U.S. Pat. No. 6,156,009 both to Grabeck may be used to access the pericardial space. In addition, devices as disclosed in U.S. Pat. No. 5,972,013 to Schmidt, U.S. Pat. No. 5,827,216 to Igo, et al., U.S. Pat. No. 6,162,195 to Igo, et al., U.S. Pat. No. 4,991,578 to Cohen and U.S. Pat. No. 5,336,252 to Cohen may be used, for example, to access the pericardial space. These patents are incorporated herein by reference.

In one embodiment of the present invention, one or more devices may be used within the pericardial space for creating space and visualizing the surface of the heart. For example, a device comprising a rigid rod with a light may be used to push on the interior of the pericardium and to move the lung laterally if desired. Another device comprising a flat malleable spatula may be used to rotate the heart and expose the posterior lateral portion of the heart if desired. The spatula device may be bent or formed into whatever shape is required to move and rotate the heart.

In one embodiment of the present invention, a suction positioning device as described in U.S. Pat. No. 6,447,443 to Keogh et al., incorporated herein by reference, may be used to move the heart around and/or hold the pericardium out of the way. As shown in FIG. 9, the positioning device 90 may be used to engage the heart 94 and to position the heart into a non-physiological orientation.

Upon gaining access to the epicardial surface of the heart, the distal end of the delivery device is inserted through the mini-thoracotomy. The distal end of the delivery device is then placed against the surface of the heart and one or more needles are injected into the myocardium. Following delivery of one or more components of the composition, the needles are retracted. The heart may be repositioned if desired, for example, with a suction positioning device. The distal end of the delivery device may then be repositioned for additional delivery of one or more components of the composition or the delivery device may be removed from the patient. All incisions may then be closed using standard techniques. If the pleura is closed, a small tube for drainage may be left in place and removed the same day as surgery. If the pleura is open, a larger tube may be left in place for 24 hours.

In one, thoroscopic method, a patient is intubated with a double-lumen endobronchial tube that allows selective ventilation or deflation of the right and left lungs. The left lung is deflated, thereby helping to provide access to the surface of the heart. The patient is rotated approximately 30° with the left side up. The left arm is placed below and behind the patient so as not to interfere with tool manipulation during the delivery of one or more components of the composition. While port positions depend to a large extent on heart size and position, in general a 7th (seventh) and 5th (fifth) space mid (to posterior) axillary port for tools and a 3rd (third) space anterior axillary port for the scope is preferable. A variety of endoscopes or thoracoscopes may be used including a 30 degree offset viewing scope or a straight ahead viewing scope. In general, short 10 to 12 mm ports are sufficient. A soft 20 mm port with an oval cross section sometimes allows for two tools in the port without compromising patient morbidity.

The pericardium may be opened by a number of different techniques, as noted above. Upon gaining access to the epicardial surface of the heart, the distal end of the delivery device is inserted through an appropriate port. The distal end of the delivery device is then placed against the surface of the heart and one or more needles are injected into tissue.

Following delivery of one or more components of the composition, the needles are retracted. The distal end of the delivery device may then be repositioned for additional delivery of one or more components of the composition or the distal end of the delivery device may be removed from the patient. All incisions may then be closed using standard techniques. Some methods may utilize insufflation, in which the incision or port is sealed about the device shaft and the interior of the thorax pressurized.

In one, sternotomy, method, the distal end of the delivery device may be inserted through an incision made through the sternum. In yet another method, a xiphoid incision method, an incision is made below the sternum and the distal end of the delivery device is then inserted through the incision. The term “xiphoid incision” refers to a surgical incision proximate to, but not necessarily directly above, the xiphoid appendage. The xiphoid incision of the present invention provides a surgical field and access site to the heart that extends through an opening beneath the sternum and preferably immediately beneath the lowest rib.

A vertical skin incision is made above the xiphoid process and the center of the xiphoid appendage is transected. Because the xiphoid appendage is cartilaginous, the appendage does not have to be removed and the sternum does not have to be transected. The total length of the xiphoid incision depends on length of xiphoid appendage, i.e., longer xiphoids are less likely to require any cutting into the sternum. The maximum incision is preferably approximately 6-7 cm from below the tip of the xiphoid appendage upwards towards the patient's head. The incision may be extended downward below the xiphoid appendage to the extent necessary to provide an adequate surgical field, but as noted above, the maximum length should not greatly exceed 6-7 cm. The incision may be strictly vertical or may be slightly curved, following the outline of the butt of either the right or left rib cage. In most cases, a curved incision will follow the lower left rib. An approximately 1 cm incision may be made in the pericardium to accommodate insertion of a surgical scope. The scope preferably has a flexible housing and at least a 16× magnification. Insertion of the scope through the pericardial incision allows the surgeon to inspect the epicardial surface of the heart thereby allowing the physician to plan the procedure depending on the clinical status of the individual patient. At this point, the surgeon can confirm that a xiphoid access is appropriate for the particular procedure to be performed.

A vertically offsetting retractor or access platform may be used to engage a portion of the rib cage capable of lifting at least one rib and preferably more than one rib and the sternum, see U.S. Pat. No. 6,199,556 to Benetti et al. This patent is incorporated herein by reference. The term “offsetting” is used herein to describe the manipulation of at least one rib that provides access to the thoracic cavity via the xiphoid incision, generally described herein as “xiphoid access.” Typically, the vertical offsetting procedure comprises engaging the lowermost rib with an offsetting retractor or access platform and lifting at least a portion of the lowermost ribs. This may be accomplished by simultaneously applying force at one or more points about the chest and pelvis, and preferably includes at least a structural force applied vertically to orient at least a portion of the lower region of the sternum and rib cage relative to the remainder of the body below the rib cage. As noted, this orientation is most readily achieved by lifting one half of the lower edge of the rib cage, adjacent to the xiphoid appendage using a specially designed surgical retractor. Although retraction devices such as those described in U.S. Pat. No. 5,730,757 are preferred, other more conventional devices could be adapted, see for example U.S. Pat. Nos. 5,026,779, 4,726,358 and 4,852,552. These patents are incorporated herein by reference. Collectively, these devices can provide access to a beating heart via a xiphoid incision and comprise means for offset retraction of the lower rib cage

Since the size of the incision is preferably minimized in a xiphoid procedure, an organ or tissue positioner may advantageously be used to retract or reposition tissue or internal organs at the site of the incision or inside the thoracic cavity near the site of the surgery. The positioner or retractor may be of any conventional structural design, or expandable by inflation on manipulation, and is preferably suitable for minimally invasive procedures. Moreover, a tissue or organ positioner may be affixed to the offsetting retractor during the procedure to maintain access to the surgical field.

Upon gaining access to the epicardial surface of the heart, the distal end of the delivery device is inserted through the xiphoid incision. The distal end of the delivery device is then placed against the surface of the heart and one or more needles are injected into the myocardium. Following delivery of one or more components of the composition, the needles are retracted. The distal end of the delivery device may then be repositioned for additional delivery of one or more components of the composition or the distal end of the delivery device may be removed from the patient. All incisions may then be closed using standard techniques. A small incision may be made below the xiphoid appendage and a drainage tube may be inserted into the pericardium, if the pleura has not been and into the pleura itself if it has been opened. Before finally closing the xyphoid incision, a scope may be used to check the position of the drainage tube, and to check the integrity of the pleura.

In one embodiment of the present invention, passages are made through the skin into the thoracic cavity. The passages may be formed employing one-piece rods or trocars of prescribed diameters and lengths that are advanced through body tissue to form the passage and then removed so that other instruments can be advanced through the passage. The passage may also be formed employing two piece trocars that comprise a tubular outer sleeve, sometimes referred to as a port or cannula or at times as the tubular access sleeve itself, having a sleeve access lumen extending between lumen end openings at the sleeve proximal end and sleeve distal end, and an inner puncture core or rod that fits within the sleeve access lumen. The inner puncture rod typically has a tissue penetrating distal end that extends distally from the sleeve distal end when the inner puncture rod is fitted into the sleeve access lumen for use. The two-piece trocar can be assembled and advanced as a unit through body tissue, and the inner puncture rod then removed leaving the tubular access sleeve in place to maintain a fixed diameter passage through the tissue for use by other instruments.

In one of these ways, a tubular access sleeve is placed through a passage that is made as described above in the chest wall of a patient between the patient's 2nd (second) rib and 6th (sixth) rib, for example. The selection of the exact location of the passage is dependent upon a patient's particular anatomy. A further conventional tubular access sleeve is placed in a different passage that is also made as described above in the chest wall of patient.

In one embodiment of the present invention, the patient's left lung is deflated to allow unobstructed observation of the pericardium employing a thoracoscope or other imaging device inserted through a sleeve lumen of a tubular access sleeve. The thoracoscope or other imaging device may have its own light source for illuminating the surgical field. Deflation of the patient's lung may be accomplished by use of a double lumen endotracheal tube that is inserted into the trachea, and independent ventilation of the right, left or both lungs can be selected. The left lung will collapse for visualization of the structures of the left hemi-sternum when ventilation of the left lung is halted and the left thoracic negative pressure is relieved through a lumen of the tubular access sleeve or a further access sleeve to atmospheric pressure. After deflation, the thoracic cavity may be suffused with a gas, e.g., carbon dioxide, introduced through a lumen of the tubular access sleeve or the further access sleeve to pressurize the cavity to keep it open and sterile. The pressurized gas keeps the deflated lung away from the left heart so that the left heart can be viewed and accessed and provides a working space for the manipulation of the tools of the present invention. It will be understood that the access sleeve lumens must be sealed with seals about instruments introduced through the lumens if pressurization is to be maintained

A thoracoscope can then inserted into the lumen of a tubular access sleeve to permit wide angle observation of the thoracic cavity by a surgeon directly through an eyepiece or indirectly through incorporation of a miniaturized image capture device, e.g., a digital camera, at the distal end of the thoracoscope or optically coupled to the eyepiece that is in turn coupled to an external video monitor. The thoracoscope may also incorporate a light source for illuminating the cavity with visible light so that the epicardial surface can be seen directly indirectly. The thoracoscope may be used to directly visualize the thoracic cavity and obtain a left lateral view of the pericardial sac or pericardium over the heart.

The elongated access sleeve provides an access sleeve lumen enabling introduction of the distal end of a pericardial access tool. The tubular access sleeve and the pericardial access tool are employed to access the pericardial space and epicardium surrounding the heart. The distal end of the delivery device is then advanced through the elongated access sleeve, through the incision formed through the pericardium and placed against the epicardium. One or more needles of the delivery device are then advanced into the myocardium and one or more components of the composition are then delivered into the myocardium. The one or more needles can comprise any of the needles described herein.

When practicing the current invention, a clinician may need to procedurally stabilize a beating heart for injection. Significant motion of the heart during the cardiac cycle poses a challenge when attempting to deliver composition to the myocardium in a temporally and spatially controlled fashion. In one embodiment of the invention, the heart can be manually stabilized by the clinician or an assistant simply holding it in her hand so that it will remain in one location relevant to the delivery device. Other embodiments of the current invention can achieve this procedural stabilization by pharmacologic or electrophysiologic means. Regardless of the method used, the goal is to place a heart in controlled intermittent asystole. In at least one embodiment of the present invention, a heart is procedurally stabilized using pharmacologic asystole. In other embodiments, a heart is procedurally stabilized using electrophysiologic over-drive pacing or other algorithms that render the heart fairly static. These include reversible initiation of asystole, fibrillation, or a prolonged refractory state.

In other embodiments of the present invention, areas of a beating heart may be stabilized structurally. In one embodiment of the present invention, a tissue stabilizer device, (e.g., the Medtronic Octopus® device) may be used to procedurally stabilize the immediate area of the treatment site during an epicardial delivery procedure. In another embodiment, the immediate area of the treatment site is procedurally stabilized using a tissue stabilizing member or device that is part of the delivery device. Other embodiments of the invention use other structural means such as sleeves and compressors that are held against the treatment site to provide procedural stabilization of the heart around the delivery area.

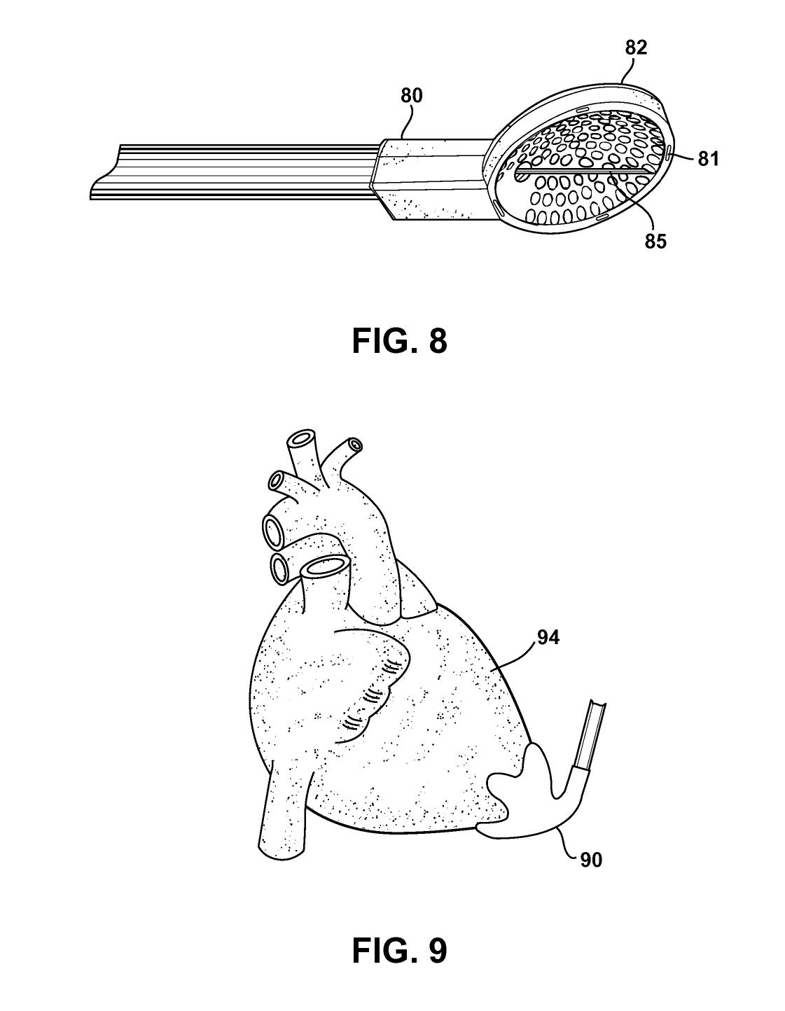

FIG. 8 depicts an example of a device for stabilizing the myocardium, relative to an delivery device, when a composition is being delivered into the myocardium. The device 80 is a suction tool that can be placed on the myocardium. When suction is applied via a lumen in the device through a plurality of suction ports 81, a portion of the myocardium will be drawn up into the dome shaped suction area 82 where it will be temporarily stabilized relative to an injection needle 85. This type of device allows a clinician to deliver the compositions described herein to a beating heart or to a temporarily non-beating heart.

One method to predictably deliver compositions into such a moving target tissue is to time injections specifically for delivery during a select portion of the cardiac cycle. In one embodiment of the present invention, one or more electrodes may be used as stimulation electrodes, e.g., to pace the heart during delivery of composition. In this way, the cardiac cycle is made to be predictable and injection can be timed and synchronized to it. In fact, the beat-to-beat period can be artificially lengthened so as to permit complete delivery during a specific (and relatively) stationary phase of the cardiac cycle. In one embodiment, the delivery device includes one or more stimulation and/or sensing electrodes. In one embodiment of the present invention, sensors may be used to sense contractions of the heart, thereby allowing the delivery of composition to be timed with cardiac contractions. For example, it may be desirable to deliver one or more components of the composition between contractions of the heart.

Cardiac contraction sensors may be any suitable sensor, e.g., an electrical sensor, a chemical sensor or a biosensor, for detecting one or more signals indicative of a cardiac contraction or heartbeat. In one embodiment, the delivery device may include one or more cardiac contraction sensors. In one embodiment, a sensor may be used to monitor the electrical activity of the heart by picking up and amplifying electrical signals from the heart and displaying a visual output and/or providing an audio output. For example, the output may be displayed on a display interface. The surgeon may check this output to determine the optimal time to inject the needles and/or composition into the tissue.

A cardiac contraction sensor may be a sensor that detects cardiac depolarizations. The electrical signal generated by the sinus node of the heart causes the atria to contract to force blood into the ventricles. After a brief delay, the ventricles contract to force blood out through the body. The contraction of the ventricles is reflected by the passage of a depolarization wavefront through the heart muscle. If a depolarization is sensed, a beat is likely to occur. One such depolarization sensor is disclosed in U.S. Pat. No. 5,156,149 entitled “Sensor for Detecting Cardiac Depolarizations Particularly Adapted for use in a Cardiac Pacemaker”, Oct. 2, 1992, to inventor Hurdlik. This patent is assigned to Medtronic, Inc. and is incorporated herein by reference.

A cardiac contraction sensor may be coupled to a cardiac stimulator. A cardiac contraction sensor may be an apparatus that senses power levels of depolarizations in heart tissue. Such a sensor may be used to distinguish between normally conducted and ectopic heart beats while the heart is beating or may be used to sense an imminent heart beat while the heart is slowed or substantially stilled during a medical procedure. One apparatus that may serve as such a sensor is disclosed in U.S. Pat. No. 5,411,529 entitled “Waveform Discriminator for Cardiac Stimulation Devices”, May 2, 1995, to inventor Hurdlik. This patent is assigned to Medtronic, Inc. and is incorporated herein by reference. Other suitable sensors may also serve as cardiac contraction sensor.

A variety of methods are disclosed to hold the heart stationary or relatively stationary in order to facilitate controlled delivery of compositions to specific target sites within the myocardium. These include minimally invasive pharmacologic methods (utilization of specific drugs such as adenosine), slightly more invasive electrophysiologic methods and invasive methods (Octopus®/Starfish® devices) as described herein.

In one embodiment of the present invention, a nerve stimulator may be used to electrically manipulate cardiac rhythm by stimulating the vagus nerve. This vagal stimulation may produce asystole (slowing or stopping of the heart's beating.) Once this induced asystole is stopped, i.e., once the vagal stimulation is stopped, the heart may be allowed to return to its usual cardiac rhythm. Alternatively, the heart may be paced, thereby maintaining a normal cardiac output. Vagal stimulation, alone or in combination with electrical pacing, may be used selectively and intermittently to allow a physician to perform delivery of one or more components of the composition into a temporarily stopped heart. For example, stimulation of the vagus nerve in order to temporarily and intermittently slow or stop the heart is described in U.S. Pat. Nos. 6,006,134, 6,449,507, 6,487,449, 6,532,388, and 6,628,987. These patents are assigned to Medtronic, Inc. and are incorporated herein by reference.

In one embodiment of the present invention, a patient's heart may be engaged and positioned using a tissue positioner, as described earlier. Once the heart is in a desired orientation, a nerve that controls the beating of the heart is stimulated to slow down or stop the contractions of the heart. Such a nerve may be for example a vagal nerve. During this time, one or more of a variety of pharmacological agents or drugs may be delivered to the patient. These drugs may produce reversible asystole of a heart while maintaining the ability of the heart to be electrically paced. Other drugs may be administered for a variety of functions and purposes as described above. Drugs may be administered at the beginning of the procedure, intermittently during the procedure, continuously during the procedure or following the procedure.

Typically, vagal nerve stimulation prevents the heart from contracting. This non-contraction must then be followed by periods without vagal nerve stimulation during which the heart is allowed to contract, and blood flow is restored throughout the body. Following initial slowing or stopping of the heart, one or more components of the composition may be delivered via a delivery device of the present invention to the stopped or slowed heart. Following a brief interval of nerve stimulation while the injection is performed, nerve stimulation is ceased and the heart is allowed to contract. A cardiac stimulator or pacemaker may be used to cause the heart to contract or the heart may be free to beat on its own. In one embodiment of the present invention, one or more electrodes may be used for pacing the heart as desired. A processor may control both cardiac and nerve stimulation. For example, a processor may cease nerve stimulation and automatically begin cardiac stimulation. Following injection, the heart may be repositioned if necessary or desired.

In one embodiment of the present invention, a patient's beating or stopped heart may be engaged and positioned by a tissue positioner, as described earlier, to provide access to the posterior or backside of the heart, for example. The tissue positioning device may be inserted into a patient through a percutaneous opening. For example, the positioning device may be positioned through a sternotomy or thoracotomy or through a xiphoid incision as described above. Heart positioning may occur throughout the entire procedure in a continuous or intermittent manner. Upon completion of one or more injections of compositions at a first location, the heart may be repositioned to provide better access for additional injections of compositions at additional locations.

Regardless of the method used to access a heart having a region of injured myocardium or stabilize the heart, the delivery devices used may need to be capable of injecting multiple components separately into the myocardium. One embodiment of the current invention enables repeated injection by a single device. This may be achieved by a proximal one-hand trigger that enables predictable delivery of a determinable (e.g., dial-in) dose of a single- or multiple-constituent composition in a determinable ratio. A different embodiment of the current invention utilizes delivery devices having dual lumen needles/delivery catheters, and at least one other embodiment uses delivery devices having three or more lumen needles/delivery catheters. The lumens in the needles/delivery catheters can be in a coaxial configuration or a biaxial configuration.

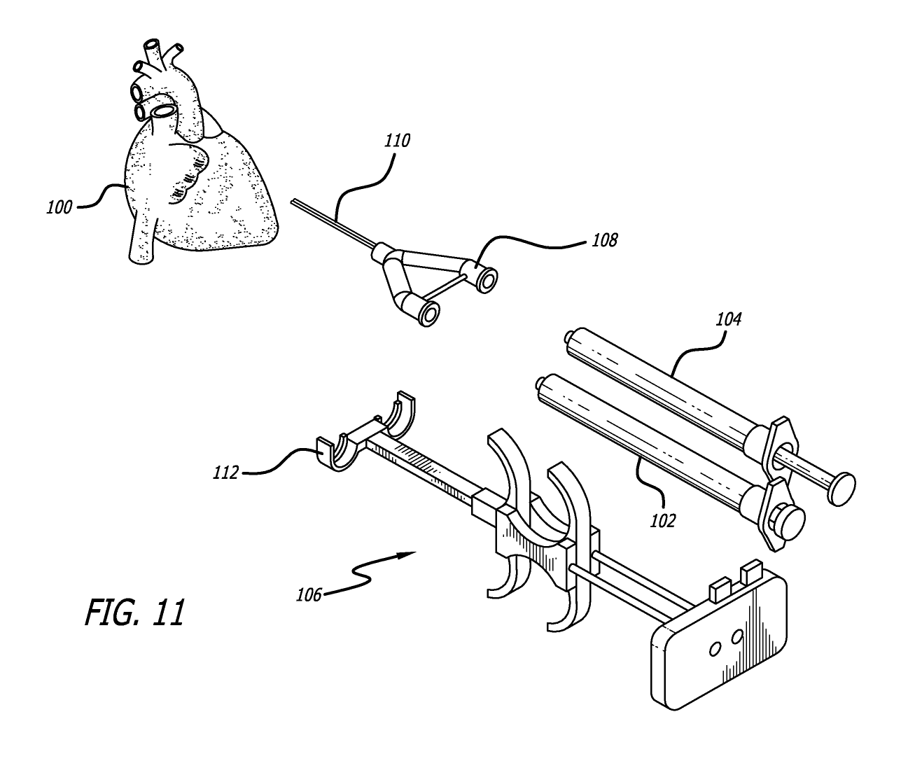

At least one embodiment of the present invention includes two or more side-by-side syringes for one-handed injection of the multiple composition components. In one embodiment, the device of FIG. 11 is used to inject a multi-component composition into an injured heart 100. In the embodiment of FIG. 11, two components of the composition of the present invention are housed separately in syringes 102 and 104. Syringes 102 and 104 are disposed in cradle 112 within a handle assembly 106 to allow one-handed injection of the composition. An adapter 108 couples to the syringes 102 and 104 to a biaxial needle 110. Biaxial needle 110 allows the delivery of two components of a composition, in a non-limiting example, PRP and thrombin, to a treatment site in heart 100.

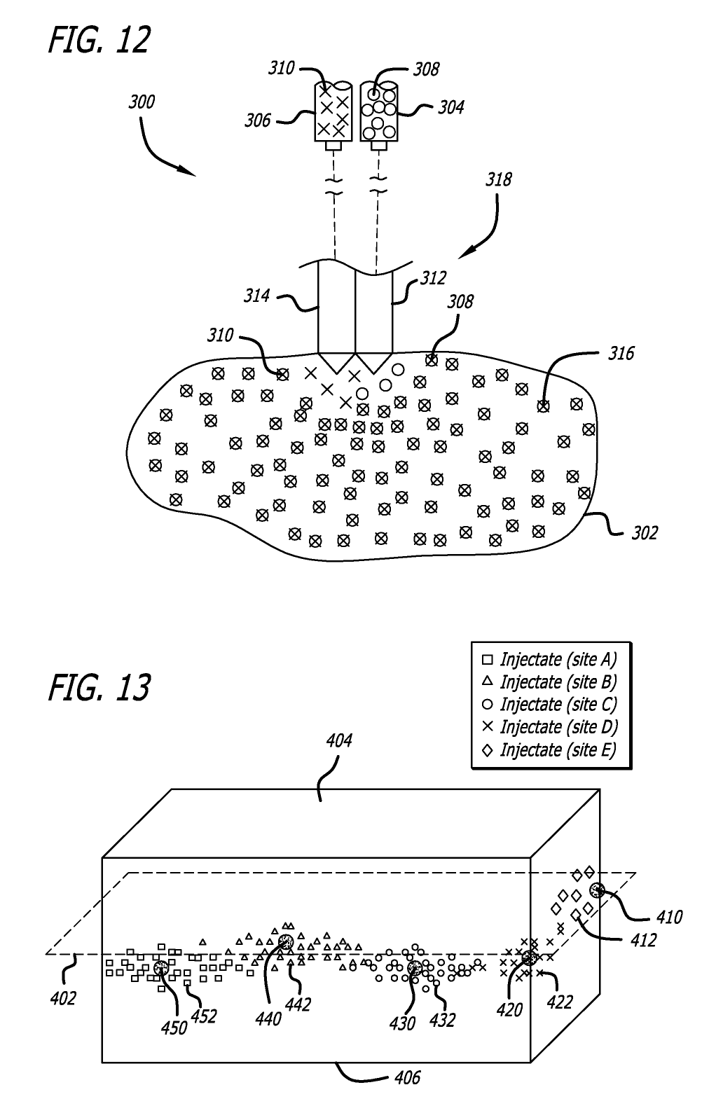

FIG. 12 represents an enlarged view of the injection of a two-component composition according to the present invention using a biaxial injection needle containing delivery device 300. Component 310 is held in reservoir or syringe 306 and component 308 is held in reservoir or syringe 304. Components 310 and 308 are caused to pass into biaxial needle 318 comprising needle lumen 314 for injection of component 310 and needle lumen 312 for injection of component 308. Components 310 and 308 are injected into the treatment site 302 simultaneously and the two components combine to form composition 316. Immediately after injection, components 310 and 308, and to a certain extent composition 316 diffuse through the tissue at treatment site 302. The components and compositions have been observed to diffuse up to two centimeters in myocardial tissue (see also FIG. 13)[

The delivery system may delivery the components of the composition in a prescribed ratio. This ratio may be pre-set (and fixed) or dialable (and dynamic). One embodiment of the present invention utilizes separate gears or levers (with gear-ratio or lever-ratio that are settable) to enable delivery of multiple compounds in different ratios without generating a pressure gradient between syringes. Other multi component delivery devices of the current invention include lumens of different caliber to allow for pre-determined ratio of each component. Some multi-component delivery devices of the current invention include lumens of different lengths, such that one component is released more distally than another. Still other devices incorporate one or more mixing chambers in the device. At least one embodiment of delivery devices of the current invention includes single lumen needle/catheters that are used for serial delivery of multiple components (one after another).

Several embodiments of delivery devices can be placed in a vessel neighboring the target treatment site and used to deliver compositions to the myocardium by piercing through the vessel wall and navigating to the desired location with the needle-tip or a microcatheter that is contained in the needle. The catheter or needle may contain a local imaging system for identifying the target area and proper positioning of the delivery device. The device may include one or more needles having a closed distal tip and one or more side openings for directing a substance substantially laterally from the distal tip into the myocardium. Preferably, the needle has a sufficiently small gauge diameter such that the needle track in the myocardium is substantially self-sealing to prevent escape of the composition upon removal of the needle. Recent data (obtained in the context of epicardial delivery) demonstrated hemostasis in vivo when platelet gel was injected through even a large 18 gauge injection needle. This result could be attributable to the rapid coagulation achieved by the components injected and the inherent hemostatic properties of platelet gel. In another embodiment, the needle gauge is smaller than 18 gauge. In one embodiment, the needle gauge is 26 gauge.

Alternatively, the delivery assembly may include one or more needles having a plurality of lumens that extend between a multiple line manifold on the proximal end to adjacent outlet ports. A multi-lumen needle assembly may allow components of a substance to be independently injected, thereby allowing the components to react with one another following delivery within the selected tissue region, as described herein.

In one embodiment, a multi lumen needle assembly may allow two components of a composition to be simultaneously, independently injected, which may then react with one another once within the selected tissue region, as described herein. In another embodiment having a multi lumen needle assembly, the lumens empty into a mixing chamber located near the distal tip of the needle and the components of the injected substance are mixed with each other immediately prior to being injected into the selected tissue region.

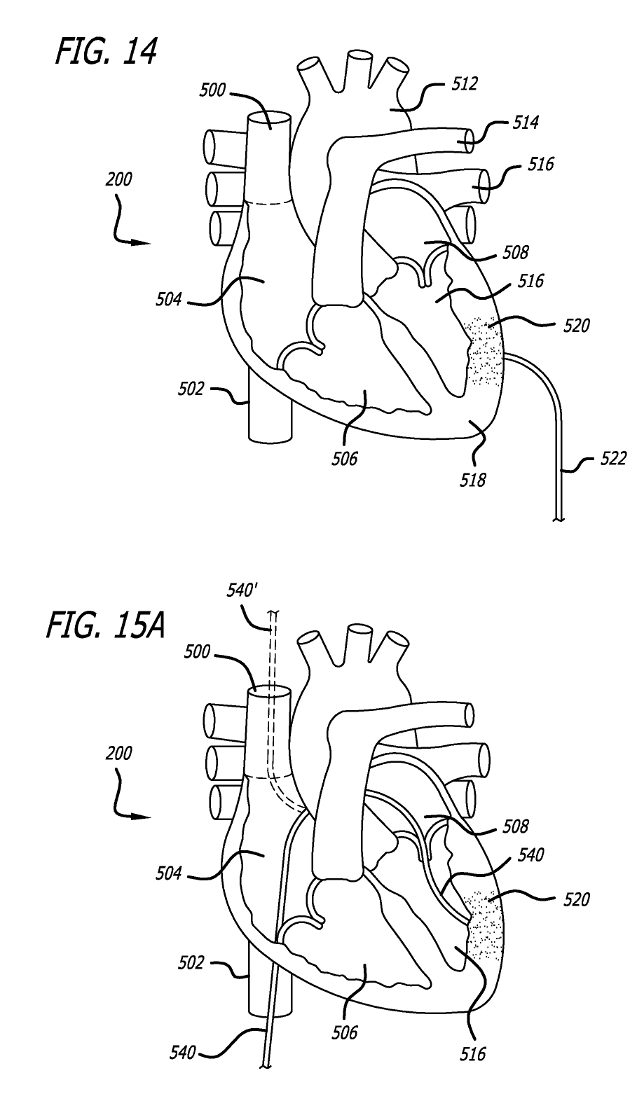

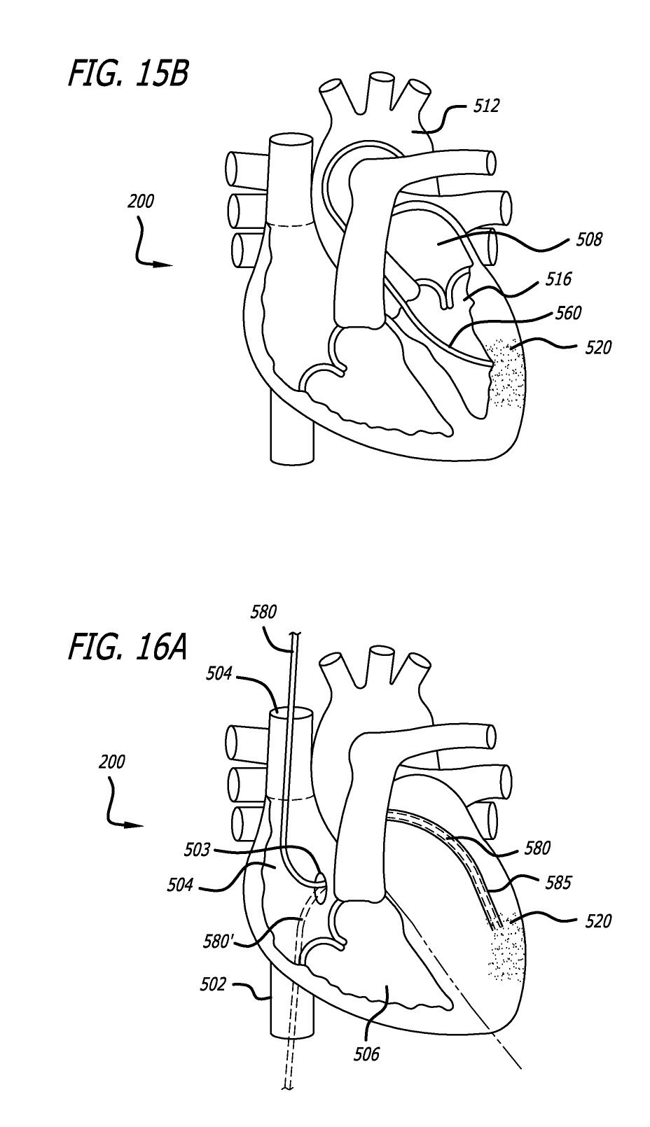

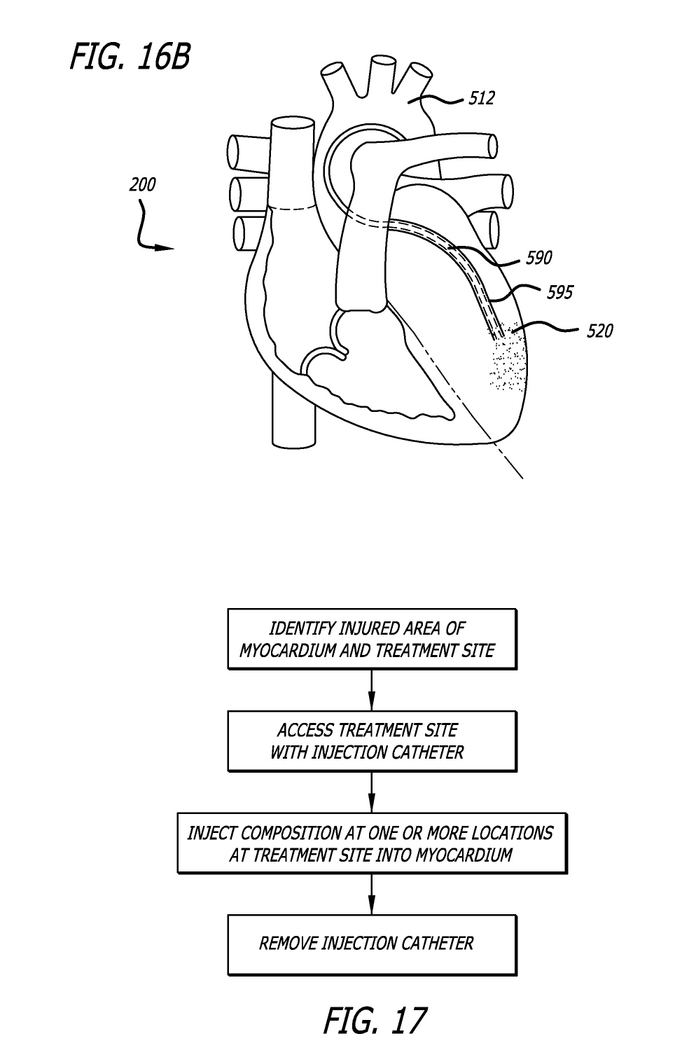

Compositions of the current invention can be delivered to the myocardium by a catheter system. Catheter delivery systems suitable for the current invention include systems having multiple biaxial or coaxial lumens with staggered or flush tips. The catheter systems of the current invention can include needles or other injection devices located at the distal end, and syringes at the proximal end of the catheters. The catheters and other delivery devices of the current invention can have differently sized lumens to ensure that multi-component compositions can be delivered to the myocardium in the desired ratio. Another embodiment of a catheter system may be used to create a composition reservoir within the myocardium itself to provide sustained delivery. A catheter may be introduced endovascularly into a blood vessel until the distal portion is adjacent the desired treatment location. The needle assembly may be oriented and deployed to puncture the wall of the vessel and enter the myocardium. The composition can then be injected into the myocardium and, thereby, form a reservoir. When catheter systems are used, a clinician can navigate to a patient's heart using one of the plurality of routes known for accessing the heart through the vasculature, or navigation to a heart chamber for delivery of the compositions epicardially (FIG. 14), endocardially (FIG. 15A-B) or transvascularly (FIG. 16A-B). In FIGS. 14 and 15 the entire heart is shown in cross section. In FIG. 16 the right ventricle and atrium are shown in cross-section while the left ventricle and atrium are shown closed with the epicardial surface and its coronary vessels in view

Epicardial delivery of compositions comprises accessing a treatment site 520, in a non-limiting example, in the left ventricle 516 of a heart 200 from the epicardial, that is, exterior, surface of the heart as depicted in FIG. 14 and injecting the composition into treatment site 520 with a delivery device 522.

Endocardial delivery of compositions (FIGS. 15A and B) comprises accessing a treatment site 520, for example, in the left ventricle of a heart 200, with a delivery device 540, 540′ percutaneously through an anterograde approach (FIG. 15A) through the superior vena cava 500 (delivery device 540′) or inferior vena cava 502 (delivery device 540) into the right ventricle 504. The delivery device 540 is passed through the interatrial septum into the left atrium 508 and then into the left ventricle 516 to reach treatment site 520 where the composition is injected with delivery device 540. An alternative endocardial delivery method depicted in FIG. 15B comprises accessing a treatment site 520, for example, in the left ventricle of a heart 200, with a delivery device 560 percutaneously through a retrograde approach through the aorta 512 into the left atrium 508 and then into the left ventricle 516 to reach treatment site 520 where the composition is injected with delivery device 560.

Transvascular delivery of compositions (FIGS. 16A and B) comprises accessing a treatment site 520, for example, in the left ventricle of a heart 200, with delivery device 580, 580′ percutaneously through a venous approach (FIG. 16A) through the superior vena cava 500 (delivery device 580) or inferior vena cava 502 (delivery device 580′) into the right ventricle 504. The delivery device 580 is passed through the coronary sinus 503 into the cardiac venous system via these veins and, if needed, leaving these veins by tracking through myocardial tissue, it reaches treatment site 520 where the composition is injected with delivery device 580. An alternative transvascular delivery method depicted in FIG. 16B comprises accessing a treatment site 520, for example, in the left ventricle of a heart 200, with a delivery device 590 percutaneously through an arterial approach through the aorta 512 into a coronary artery 595 to reach treatment site 520 where the composition is injected with delivery device 590.

Devices for injecting the compositions of the current invention can include refrigerated parts for keeping the various components of the compositions cool. Various embodiments of delivery devices for practicing the current invention can include a refrigerated/cooled chamber for thrombin refill, a refrigerated/cooled chamber for thrombin, and/or an agitator mechanism in a PRP refill or injection chamber to prevent settling of the PRP. Delivery devices can include heating or cooling devices used to heat or cool the myocardium or compositions to speed up or slow down the gelling/hardening time after delivery. Some devices of the present invention can include catheters or other delivery devices with a cooled lumen or lumens for keeping components of the injected compositions cool while they are traveling through a device lumen. As noted above, some devices can include a mixing chamber for mixing the components of an injected composition before the substance is delivered into the tissue. In one embodiment of the invention, the PRP is stored in an agitating/vibrating chamber that provides sufficient agitation to keep the PRP homogeneous. In another embodiment, the clinician provides sufficient agitation to the delivery device by tilting, or otherwise manipulating the device to keep the PRP homogeneous.

A clinician practicing the current invention may need to make multiple injections using a single delivery assembly. Thus, at least one embodiment of the delivery devices of the current invention includes a device having at least one reusable needle. Some embodiments of the present invention may include delivery devices having an automated dosing system, e.g., a syringe advancing system. The automated dosing system may allow each dose to be pre-determined and dialed in (can be variable or fixed), e.g., a screw-type setting system. One embodiment of the current invention may include a proximal handle wherein each time the proximal handle is pushed; a pre-determined dose is delivered at a pre-determined or manually-controllable rate.

In further alternative embodiments, the delivery system may include a plurality of needle assemblies (similar to the individual needle assemblies described above), to be deployed in a predetermined arrangement along the periphery of a catheter. In one embodiment, the needle assemblies may be arranged in one or more rows. In particular, it may desirable to access an extended remote tissue region, for example extending substantially parallel to a vessel, within the myocardium. With a multiple needle transvascular catheter system, a single device may be delivered into a vessel and oriented. The array of needles may be sequentially or simultaneously deployed to inject a composition into the extended tissue region, thereby providing a selected trajectory pattern. Catheter based devices such as those described above are disclosed in U.S. Pat. No. 6,283,951, the disclosure of which is incorporated herein by reference thereto.

If a clinician is practicing the current invention using a minimally invasive or percutaneous technique, he/she may need some sort of real-time visualization or navigation to ensure site-specific injections. Thus, at least one embodiment of the present invention uses MNav technologies to superimpose pre-operative MRI or CT images onto fluoroscopic images of a delivery catheter to track it in real-time to target sites. In one embodiment, the clinician uses a contrast agent and/or navigation technologies to track the needle-tip during injection in a virtual 3-D environment. This technique marks previous injections to ensure proper spacing of future injections.

The needle assembly (or other device component) may include a feedback element or sensor for measuring a physiological condition to guide delivery of compositions to the desired location. For example, an EKG lead may be included on the distal tip or otherwise delivered within the selected tissue region to detect and guide injection towards electrically silent or quiet areas of myocardium, or to allow electrical events within the heart to be monitored during delivery of the composition. During treatment, for example, the composition may be delivered into a tissue region until a desired condition is met. Also, local EKG monitoring can be used to target and guide injection towards electrically silent or quiet areas of myocardium.

Regardless of the device used to deliver the composition or how the clinician accesses the myocardium, a clinician practicing the current invention may have the need for precise local placement and depth-control for each injection. In one embodiment of the present invention, the substance is delivered/injected to a depth in the myocardium that is approximately midway between the outside wall and the inside wall of the myocardium. In other embodiments, the substances are delivered to a depth that is closer to either the inside wall or the outside wall. The substances may be delivered intra-myocardially, sub-endocardially, or sub-epicardially. In another embodiment of the invention, the depth of the injection will vary based on the thickness of the target tissue and the depth is less at the apex of a heart than it is at other locations on the heart.

To achieve depth control, the delivery device of at least one embodiment of the present invention includes a stopper fixed (or adjustably fixed) on the needle shaft, at a desired distance from needle's distal tip, to prevent penetration into tissue beyond a specified depth. Some embodiments use the method of injecting one or more needles into tissue at a tangent to the tissue surface to control the depth of the injection. In at least one embodiment of the present invention, the needle can be positioned to inject at an angle perpendicular (90 degrees) to the tissue, tangential (0 degrees) to the tissue, or any desired angle in between. Suction can facilitate controlled positioning and entry of the injector.

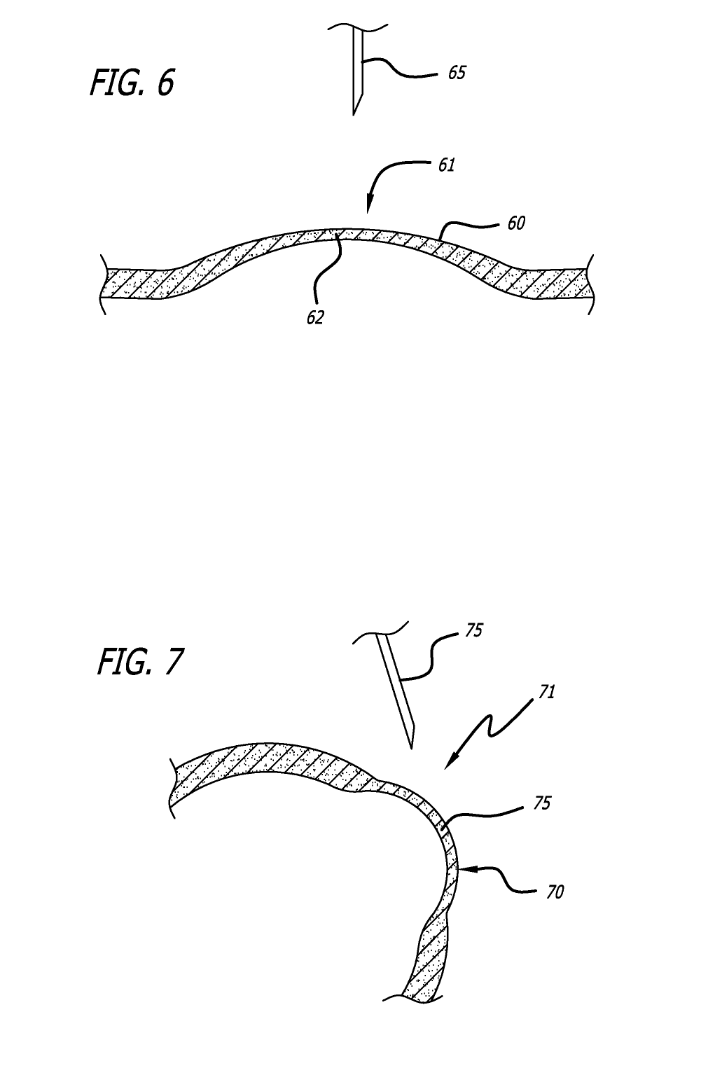

Referring now to FIG. 6, there can be seen an example of an injection according to one embodiment of the current invention wherein the needle 65 of delivery device (not shown) is approaching at an angle generally perpendicular to a remodeled portion of myocardium 60. The needle will puncture the myocardium at a point 61 directly above the desired delivery location 62 within the myocardium. The device may include one or more means, for example, as described above, to ensure that the needle achieves the desired penetration depth into the myocardium.

FIG. 7 shows an example of an injection according to one embodiment of the current invention wherein the needle 75 of the delivery device (not shown) is approaching at an angle approximately tangentially to the desired injection point 71 of a myocardium. The needle will puncture the myocardium at a point 71 located a desired distance tangentially from the desired delivery location 72 within the myocardium. Although not shown in the figure, a suction type stabilizer device, as described above (an example of this type of device is shown in FIG. 8), may be applied to the surface of the heart, at a location around or near the injection site, to stabilize the target region or the adjacent beating heart, respectively. The device will secure a generally dome shaped section of myocardium 70 therein so that the composition can be delivered. The device can include one or more means as described above to ensure that the needle achieves, but does not exceed, the desired penetration into the myocardium.

At least one embodiment of the present invention uses a “Smart-Needle” to detect distance from the needle tip to the ventricular blood compartment or endocardial surface, so that the needle tip is maintained in the cardiac wall. Such a needle can rely on imaging around or ahead of the needle tip by imaging modes such as ultrasound.

At times it might be desirable to distribute the composition as widely as possible around the injection site. It might also be desirable to have the composition be uniformly distributed around the injection site. One method for enhancing distribution of a composition around an injection site is to use needles having holes in the side vs. using needles having holes in the end. Multiple side holes can provide a wider distribution of composition around the injection site. Side holes also provide access to the tissue from a multitude of places rather than just from the end of the needle, thereby requiring less travel of the composition for wider distribution. A potential benefit of side holes in the needles is that if the needle tip accidentally penetrates through the heart wall and into a cardiac chamber, the composition may still be injected into cardiac tissue as opposed to being injected into the blood stream within the cardiac chamber. Another method for enhancing distribution of a composition around an injection site is to increase the number of needles used at the injection site. If desired, the multi-needle delivery device of the present invention, allows for multiple needles to be placed close to each other in order to provide a uniform distribution over a larger area as compared to the use of a single needle device. The combination of side holes on the needles of a multi-needle device may provide a broad distribution of composition around an injection site.

In one embodiment of the present invention, suction may be used to improve the distribution of composition around the injection site. The use of suction can create a negative pressure in the interstitial space. This negative pressure within the interstitial space can help the composition to travel farther and more freely, since the composition is driven by a negative pressure gradient. The combination of suction and side holes on the needles of a multi-needle device may provide a more thorough and broad distribution of composition around an injection site.

In one embodiment of the present invention, the delivery of compositions from the delivery device into tissue may be enhanced via the application of an electric current, for example via iontophoresis. In general, the delivery of ionized agents into tissue may be enhanced via a small current applied across two electrodes. Positive ions may be introduced into the tissue from the positive pole, or negative ions from the negative pole. The use of iontophoresis may markedly facilitate the transport of certain ionized agents through tissue.

In one embodiment, one or more needles of the delivery device may act as the positive and/or negative poles. For example, a grounding electrode may be used in combination with a needle electrode via a monopolar arrangement to deliver an ionized composition iontophoretically to the target tissue. In one embodiment, a composition may be first dispersed from the needle into tissue. Following delivery, the composition may be iontophoretically driven deeper into the tissue via the application of an electric current. In one embodiment, a delivery device having multiple needles may comprise both the positive and negative poles via a bipolar arrangement. Further, in one embodiment, multiple needle electrodes may be used simultaneously or sequentially to inject a substance and/or deliver an electric current.

When practicing the current invention, one goal is to inject a substance into the myocardium while avoiding accidental delivery into one or more chambers of the heart, the coronary artery or venous system. Delivery into one or more of these areas may have negative consequences such as pulmonary or systemic embolization, stroke, cardiac congestion, and/or distant thromboembolism, for example. The current invention addresses and attempts to prevent these negative consequences in a variety of ways. In at least one embodiment of the present invention, the ratio of the components of the composition is selected so that the composition gels or polymerizes almost immediately in-situ to minimize migration of one or more of the components. In one embodiment, a balloon catheter is placed in the coronary sinus and inflated during delivery until gelling is complete. This would prevent liquid components from traveling from the tissue to the coronary venous tree and instead promote residence and gelling in the target tissue. At least one embodiment includes a pressure control system on the delivery device, to ensure that injectate pressure never exceeds ventricular chamber pressure. This would encourage retention in tissue and prevent pressure-driven migration of the composition through the thebesian venous system into the cardiac chamber. One embodiment of the present invention uses a “Smart Needle” as described above to prevent negative consequences from occurring.

At least one embodiment of the present invention includes a proximally-hand-operated distal sleeve that covers the needle tip or applies local negative pressure to prevent outward flow of component(s) from the tip of the needle between injections where multiple injections are required. In at least one embodiment, the column of components in a catheter is held under a constant minimum pressure that prevents outflow in between injections. In at least one embodiment, one-way valves may be placed within each line to prevent entry of one component into a line containing another. This is especially important when the gelling reaction is rapid and the different components need to be maintained separately until the time and site of injection. This will prevent clogging of the delivery device, which will allow repeated injections using a single device.

At least one embodiment of the present invention prevents backbleed out of the needle track, during and after removal of the needle, by keeping the needle in place for several seconds (e.g. 5-30 sec beyond the expected clotting time) following injection, to utilize the injectate as a ‘plug’ preventing back-bleed, before removing needle. In at least one embodiment of the current invention, the needle is left in place for the expected gelling time of the injected substance and then withdrawn. In one embodiment of the invention, the gelling time of an injected composition is five seconds.

Several embodiments of the current invention can include sensors and other means to assist in directing the delivery device to a desired location, ensuring that the injections occur at a desired depth, ensuring the delivery device is at the treatment site, ensuring that the desired volume of composition is delivered, and other functions that may require some type of sensor or imaging means to be used. For example, real-time recording of electrical activity (e.g., EKG), pH, oxygenation, metabolites such as lactic acid, CO₂, or other local indicators of myocardial viability or activity can be used to help guide the injections to the desired location. In some embodiments of the present invention, the delivery device may include one or more sensors. For example, the sensors may be one or more electrical sensors, fiber optic sensors, chemical sensors, imaging sensors, structural sensors and/or proximity sensors that measure conductance. In one embodiment, the sensors may be tissue depth sensors for determining the depth of tissue adjacent the delivery device. In one embodiment, a sensor that detects pH, oxygenation, a blood metabolite, a tissue metabolite, etc may be used at the end of the delivery device to alert the user if and when the tip has entered the chamber blood. This would cause the operator to re-position the delivery instrument before delivering the composition. The one or more depth sensors may be used to control the depth of needle penetration into the tissue. In this way, the needle penetration depth can be controlled, for example, according to the thickness of tissue, e.g., tissue of a heart chamber wall. In some embodiments, sensors may be positioned or located on one or more needles of the delivery device. In some embodiments, sensors may be positioned or located on one or more tissue-contacting surfaces of the delivery device. In other embodiments of the present invention, the delivery device may include one or more indicators. For example, a variety of indicators, e.g., visual or audible, may be used to indicate to the physician that the desired tissue depth has been achieved.

In some embodiments, the sensors may sense and/or monitor such things as temperature, vibration, voltage, amperage, wattage and/or impedance. The sensors may be any suitable blood gas sensor for measuring the concentration or saturation of a gas in the blood stream. For example, a sensor for measuring the concentration or saturation of oxygen or carbon dioxide in the blood and/or tissues may be employed. The sensors may be any suitable sensor for measuring blood pressure or flow, for example a Doppler ultrasound sensor system, or a sensor for measuring hematocrit levels. The sensors of may be a biosensor comprising an immobilized biocatalyst, enzyme, immunoglobulin, bacterial, mammalian or plant tissue, cell and/or subcellular fraction of a cell. For example, the tip of a biosensor may comprise a mitochondrial fraction of a cell, thereby providing the sensor with a specific biocatalytic activity.

The sensors may be based on potentiometric technology or fiber optic technology. For example, the sensor may comprise a potentiometric or fiber optic transducer. An optical sensor may be based on either an absorbance or fluorescence measurement and may include an ultraviolet, a visible or an infrared light source.

The sensors may be used to detect naturally detectable properties representative of one or more characteristics, e.g., chemical, physical or physiological, of a patient's bodily tissues or fluids. For example, naturally detectable properties of patient's bodily tissues or fluids may include pH, fluid flow, electrical current, impedance, temperature, pressure, components of metabolic processes, chemical concentrations, for example, the absence or presence of specific peptides, proteins, enzymes, gases, ions, etc.

The sensors may include one or more imaging systems, camera systems operating in UV, visible, or IR range; electrical sensors; voltage sensors; current sensors; piezoelectric sensors; electromagnetic interference (EMI) sensors; photographic plates, polymer-metal sensors; charge-coupled devices (CCDs); photo diode arrays; chemical sensors, electrochemical sensors; pressure sensors, vibration sensors, sound wave sensors; magnetic sensors; UV light sensors; visible light sensors; IR light sensors; radiation sensors; flow sensors; temperature sensors; or any other appropriate or suitable sensor. The sensors may be powered by any suitable power source. In addition, the sensors may be coupled to any appropriate output device, for example, a LCD or CRT monitor which receives and displays information regarding the sensors. In another embodiment, the sensors are imaging sensors such as, but not limited to, an MRI coil, an ultrasound probe and a radiopaque marker.

A temperature sensor may incorporate one or more temperature-sensing elements such as, for example, thermocouples, thermisters, temperature-sensing liquid crystals, or temperature-sensing chemicals. A temperature sensor could be used, for example, to monitor tissue temperature and/or composition temperature.

The signals from one or more sensor may be amplified by a suitable amplifier before reaching an output device. The amplifier may be incorporated into an output device. Alternatively, the amplifier may be a separate device. The output device may incorporate one or more processors.

In one embodiment of the present invention, the composition delivery device may comprise one or more surgeon-controlled switches and/or valves. For example, a switch or valve may be incorporated in or on the delivery device or any other location easily and quickly accessed by the surgeon for regulation of the delivery device. The switch or valve may be, for example, a hand switch or valve, a foot switch or valve, or a voice-activated switch or valve comprising voice-recognition technologies.

A visual and/or audible signal used to alert a physician to the completion or resumption of a procedure may be incorporated into the delivery device. For example, a beeping tone or flashing light that increases in frequency as the delivery procedure ends or begins may be used.

Furthermore, the delivery device may comprise sensors to allow the surgeon or clinician to ensure the delivery device is within the heart wall rather than in the ventricle at the time of injection. Non-limiting examples of sensors which would allow determination of the location of the injector include, pressure sensors, pH sensors and sensors for dissolved gases, such as oxygen. An additional sensor that may be associated with the delivery devices suitable for use with the present invention include sensors which indicate flow of blood such as a backflow port or a backflow lumen which would inform a surgeon or clinician that the needle portion of the delivery device is in an area which has blood flow rather than within a tissue.

While the volume of composition injected may vary based on the size of the heart and the extent of structural reinforcement needed, in at least one embodiment of the present invention, 50 μL of platelet gel is injected into the myocardium per injection site. In another embodiment, 200 μL to 1000 μL of the composition is delivered per injection site. In at least one other embodiment, the volume of composition injected per injection site can vary between 100 μL and 10000 μL. In one embodiment, the clinician adjusts the injection volume, the number and spacing of injection sites, and the total volume of composition per heart to optimize clinical benefit while minimizing clinical risk.

The total injection volume per heart may be dose-dependent based on the size of the heart, the size of the ischemic region of myocardium and the desired extent of structural reinforcement of the tissue. In at least one embodiment, the total volume of composition injected into the myocardium is as much as can be accommodated by the tissue in a reasonable number of injection sites. In another embodiment, the total volume of composition injected is less than 15000 μL.

The number of injection sites per heart will be based on the size and shape of the ischemic region, the desired location of the injections, and the distance separating the injection sites. In at least one embodiment, the number of injection sites can range from 5-25 sites. The distance separating injection sites will vary based on the desired volume of platelet gel to be injected per injection site, the desired total volume to be injected, and the condition of the ischemic myocardium. In at least one embodiment, the distance between injection sites is approximately 2 cm and in at least one other embodiment, the distance between injection sites is 1 cm. In still another embodiment, the separation distance between injection sites can range between about 50 mm and about 2 cm. In another embodiment, the distance between injection sites can be in the range of 0.5 cm to 2.5 cm. In another embodiment, the distance between injection sites is greater than 2.5 cm.

FIG. 13 schematically depicts an area of injured myocardium after multiple injections of a composition of the present invention. The composition is injected into the injured myocardium approximately midway between the epicardial surface 404 and the endocardial surface 406 along the plane 402 of the ventricle or chamber. The composition is injected into multiple injection sites 410, 420, 430, 440 and 450 resulting in the diffusion of injectate several centimeters from the injection site. The injected composition diffuses such that, if multiple injections are approximately 2 cm apart, the composition forms an overlapping field of structural support material. For example, composition 412 is injected at injection site 410 and diffuses as depicted in FIG. 13. Further, composition 422 is injected at injection site 420 and diffuses and intermingles with composition 412. This is repeated at injection sites 430, 440 and 450 such that compositions 412, 422, 432, 442 and 452 form a continuous overlapping field of structural support material. In this embodiment, compositions 412, 422, 432, 442, and 452 are the same composition, in a non-limited example autologous platelet gel. In another embodiment, more than one composition can be injected into a treatment site.

The location of the delivery can vary based on the size and shape of the injured region of myocardium, and the desired extent of structural reinforcement of the tissue. In at least one embodiment of the present invention, the composition is delivered only into the injured myocardium, while in other embodiments the peri-injury zone around the injured region is treated, and, in at least one other embodiment, the composition is delivered into only the healthy tissue that borders an injured region. In other embodiments, the composition may be delivered to any combination of the regions of injured myocardium, myocardium in the peri-injury zone, and healthy myocardium.

The timing of composition delivery relative to an injurious event will be based on the severity of the injury, the extent of the injury, the condition of the patient, and the progression of any tissue remodeling. In at least one embodiment, the composition is delivered one to eight hours following an injurious event such as an MI, for example within one to eight hours following ischemia-reperfusion (in the catheterization lab setting immediately after re-perfusion). In another embodiment, the composition is delivered to the myocardium within one hour of an injurious event. In another embodiment the composition is injected three to four days after an injury (after clinical stabilization of the patient, which would make it safe for the patient to undergo a separate procedure). In at least one embodiment, the composition is delivered more than one week after the injury. Other times for injecting compositions into the myocardium are also contemplated, including prior to any injurious event, and immediately upon finding an area of injured myocardium (for preventing additional remodeling in older injuries). In another embodiment of the invention, compositions can be injected into the myocardium years after an injurious event.

In addition to the foregoing uses for the compositions, methods and systems of the present invention, it will be apparent to those skilled in the art that other injured tissues, in addition to injured myocardium, would benefit from the delivery of structural support materials to treat the injuries. Non-limiting examples of such tissues include the stomach, to reduce food intake and increase satiety; the abdominal wall, to prevent and treat hernias and the bladder to prevent or treat incontinence.

### EXAMPLES

Experiments have been conducted in laboratory conditions testing the methods and devices of the present invention disclosed herein. These include in vitro studies (described in Examples 1 and 2) in vivo studies conducted in healthy porcine tissue (Examples 3 and 4) and in vivo studies conducted in injured ovine tissue (Example 5).

### Example No. 1

Various compositions of the components for APG were tested in vitro using human blood, porcine blood, and ovine blood. One composition involved the extraction of 6 mL of PRP from 60 mL of whole blood (52.5 mL whole blood+7.5 mL anticoagulent [ACD-A, Anticoagulant Citrate Dextrose Solution A, comprising citric acid, sodium citrate and dextrose]). This PRP was combined approximately 10:1 (vol:vol) with bovine thrombin (1000 U/mL stock in 10% CaCl₂), such that mixing occured only in the targeted tissue. This was the composition tested in vivo as described below.

### Example No. 2:

The ability of fibrinogen to affect the gelling and/or physical properties of autologous platelet gel (APG) was directly tested in vitro. PRP and PPP were prepared from fresh sheep blood using the Medtronic Magellan® Platelet Separator. Autologous fibrinogen was further extracted from the resulting PPP using an ethanol precipitation method. Alternative methods such as cryoprecipitation can be used for isolation of fibrinogen. The precipitated fibrinogen was re-suspended in PRP to generate autologous fibrinogen-fortified PRP (AFFPRP). Two preparations of APG were compared from the same animal—(1) conventional APG made from PRP+1000 U/ml bovine thrombin in a 10:1 ratio and (2) fibrinogen-fortified APG made from AFFPRP+1000 U/ml bovine thrombin in a 10:1 ratio. The fibrinogen-fortified APG was noticeably firmer/harder than the conventional APG generated from the same animal's blood. This confirms the utility of fibrinogen to augment the mechanical properties of APG without reducing the gelling rate.

### Example No. 3:

It has been successfully demonstrated that intramural delivery of autologous platelet gel (APG) as two separate components (autologous PRP and bovine thrombin) that meet and clot in the tissue can be safely achieved in vivo.

Model & Access: A healthy pig model was used to test the safety and efficacy of delivery. One hundred and eighty milliliters of unheparinized blood was obtained and used to make 18 cc of PRP using a Medtronic Magellan® Autologous Platelet Separator on the day of the procedure. The animal was then heparinized to an activated clotting time (ACT) in the 250-300 range. A median sternotomy provided access to the epicardial surface of the heart.

Injections: Three injection systems were tested: System 1, a 27 gauge syringe to deliver PRP alone; System 2, an 18 gauge stainless steel needle containing a 2-lumen beveled catheter (0.0085-inch internal diameter [ID] each) with luer-lock into the needle and two independent proximal syringes (12 mL and 1 mL in size). The syringes were operated using a one-handed manifold which ensured simultaneous injection of the two components at the desired ratio (in this example, approximately 11:1). This was used to inject autologous PRP and bovine thrombin; and System 3, a suction injector which combined a suction head (to be placed on the epicardial surface of the heart) with a dual-needle injector. The suction member is driven by a vacuum pump which achieves local stabilization of the beating heart. It additionally draws the cardiac wall up into the suction cup so that the needles (entering the tissue parallel to the plane of the chamber) can be delivered at a controllable depth. The needles are driven by two separate syringes, also anchored to a one-handed injection manifold as described above as depicted in FIG. 11. A 12 mL and 1 mL syringe were used to ensure delivery of the desired ratio of autologous PRP and bovine thrombin (in this example 11:1).

Multiple injections of small volume (200-400 μl/each) were performed via an epicardial surgical approach. For injections using Systems 1 and 2 above, injections were made perpendicular to the target myocardium, and a “depth stop” was used to ensure injection to a desired depth. Target depth was 5 mm in the left ventricle and 3 mm in the right ventricle. The depth-stop consisted of a C-shaped member with a central hole through which the injection needle was passed. A side-screw (which narrows the lumen size of the depth-stop as it is screwed in) was used to anchor the depth-stop along the outside of the needle at the desired position along its length. As the needle is gently advanced into the target myocardium by the application of a force, the needle reaches the level of the depth-stop, beyond which it could not be advanced. Thus, this system ensures a fixed depth of needle penetration into tissue and ensures intramural injection occurs when wall thickness is known or estimatable.

For all injections in this study, the Medtronic Starfish® cardiac stabilizer (depicted in FIG. 9 and available from Medtronic, Inc., Minneapolis, Minn. USA) was used to provide procedural stabilization of the beating heart.

Target Tissue: Injections were performed in the left ventricle (LV, at its base, mid-position, and apex) and right ventricle (RV, at its base, mid-position, and apex). Injections into the LV were targeted to a 5 mm depth. Injections into the RV were targeted to a 3 mm depth.

Compositions: Different injectates were tested.

1) autologous PRP alone—to determine whether clot formation occurs in absence of exogenous thrombin

2) autologous PRP+bovine thrombin

3) Each of the above injections was performed with and without addition of toluidine blue dye to the autologous PRP. This was to test the utility and efficacy of a tracking dye for experimental purposes.

4) Saline control

Results: Hemostasis after APG injections was excellent. Specifically, multiple left ventricular injections of up to 1000 μl/each of APG (PRP:thrombin at 10:1) into healthy porcine myocardium were feasible and clinically safe. No adverse events were observed for up to 3 days of follow-up. Multiple right ventricular injections of up to 200 μl/each of APG (PRP:thrombin at 10:1) into healthy porcine myocardium were feasible and clinically safe. No adverse events were observed over a 2 hour follow-up period.

Twenty-three injections were well-tolerated without arrhythmia, hypoxemia, or any clinical compromise during or for 1 hr following the last injection. No thrombotic or thromboembolic sequellae were found post-mortem. All 23 injections were successful, and injection sites examined during necropsy.

Furthermore, APG injection into myocardium demonstrated a protective effect against arrhythmia. In this pig model, injection of 5600 μl of APG in divided left ventricle (LV) injections rendered the heart relatively resistant to fatal arrhythmia caused by an intravascular dose of potassium chloride (KCl). Instead of developing the expected fibrillation rhythm within 10-15 seconds of a standard dose of KCl, no arrhythmias were observed for >1.5 minutes. A second dose of KCl was required before any arrhythmias developed.

Platelet gel can be formed from PRP alone without the addition of exogenous thrombin. Platelet rich plasma injected into myocardium alone (without thrombin) surprisingly gels in situ. The present inventor has formulated the non-binding hypothesis that tissue thrombin may be present in sufficient quantities to trigger this gelling reaction. Therefore, PRP may be used to create APG within the tissue when injected alone into myocardium in vivo.

### Example No. 4:

Platelet rich plasma can be tracked in tissue by adding toluidine blue dye to the PRP. This dye does not noticably change the gelling characteristics (rate of gelling, extent of gelling, firmness of resultant gel) of PRP upon its combination with thrombin.

The pattern of APG distribution upon injection into myocardium was evaluated in vivo. In three pigs, injections of APG labeled with toluidine blue demonstrated that each injection results in distribution of the APG in all directions within the tissue. The greatest spread is along the plane of the ventricle. APG travels radially in the plane of the ventricle up to 1.5 cm. In some injections, APG was detected more than 1.5 cm away from the injection site. It is likely that APG travels during the gelling process until enough gelling has occurred to prohibit further spread of the material within the tissue.

### Example No. 5

The acute effects of APG injection into ischemic myocardium were studied in a sheep anterior infarct model. In this model, myocardial infarction results in deleterious structural and functional changes that occur within minutes of the injury. The early hallmarks of remodeling include ventricular dilatation, wall thinning, akinesis and often dyskinesis. Over time, these changes progress as remodeling continues. It was determined that early intervention post-infarction by providing APG to the injured myocardium can stunt this remodeling process.

The experiments indicated that injections were safe and well tolerated when made into infarct or non-infarct tissue, and that they can be performed safely as early as 1 hr post-MI. Controlled injections were possible with or without a cardiac stabilization device, and it was possible to make the injections without exogenous cardiac pacing. Injections were made both orthogonally and obliquely to the myocardial surface at intervals of 0.5 to 2.5 cm. The total injectate volume was tested to be safe at as high as 15.0 mL per heart, and the volume of individual injections as high as 1100 μl per injection site.

In a study of 13 sheep receiving APG one hour after infarction and followed for a 2-wk follow-up period, APG reduced arrhythmia-related post-infarction mortality. from the 25-30% seen in historical control animals receiving infarction alone to 8% in animals receiving infarction plus APG.

Remodeling was prevented acutely and at two weeks after infarction and injection of APG. In this study of 13 sheep, cardiac morphology and function were qualitatively assessed at different timepoints before and after APG injection. APG injection 1 hr post-MI resulted in a noticeable thickening of the ventricle wall, and a correction of post-MI dyskinesis acutely following injection. This effect was striking at 2 wks follow-up, when post-MI remodeling appeared to be partially or fully prevented versus historical control animals receiving infarction without APG injection.

In this anterior infarct model, the ventricle dilated to a diastolic volume of 152.4% of the pre-infarct volume within minutes of the infarction. The ejection fraction (EF) also dropped to 62.1% of baseline acutely after infarction. In five animals, APG was injected into the injured myocardium 1 hr after infarction. The treated hearts each received between 10 and 13.6 cc of APG in divided injections delivered into the myocardium. This treatment reduced the expected increase in post-infarct diastolic volume from 152.4% to 108.6% of the pre-infarct volume. This demonstrates a substantial effect of APG to prevent the expected post-MI increase in chamber volume, one of the key metrics of remodeling. In this study, APG injection also had a beneficial effect on post-MI EF, as it was restored from 62.1% to 70.3% of the pre-MI level. In one animal, APG delivery resulted in an EF that was 111.1% of pre-MI levels. That is, in this animal, EF was 45% at baseline, 35% immediately post-infarction, and 50% following administration of APG. This demonstrates that APG administration following myocardial ischemia can partially or fully reverse detrimental acute effects of infarction on EF, and in some situations may augment EF to above pre-infarct levels.

TABLE 1

| | Immediately Post-MI | Post-APG Injection |
|---|---|---|
| | (% baseline) | (% baseline) |
| Diastolic Volume | 152.4 | 108.6 |
| Ejection Fraction | 62.1 | 70.3 |

In three sheep receiving APG one hour after infarction and followed for 8 weeks, APG was surprisingly associated with neovascularization in the target ischemic tissue. This effect was not expected because the target tissue is, by definition, ischemic, and provides a poor environment for cells to survive, let alone grow to generate functional structures. In three of three animals, many small vessels were observed within the APG-treated infarct region at 8 weeks (FIG. 18). Such vessels are not usually observed in animals experiencing infarction without APG injection.

The experiments revealed that there is animal-to-animal (and presumable patient-to-patient) variability in clotting rate of APG, and (to a lesser degree) the mechanical properties of APG. Methods that demonstrate improved APG clotting rate/strength include using high-dose bovine thrombin at 1000 U/mL to make APG, and using cooled (˜0° C.) thrombin to make APG. Additionally the clotting rate/strength can be improved by fortifying autologous PRP with concentrated fibrinogen (e.g., autologous fibrinogen prepared by ethanol extraction or frozen preparation). Also, the post injection clotting rate/strength can be improved by extremely careful handling of PRP prior to injection to ensure minimal pre-activation.

Several methods were identified to enhance retention of the injectate in the target tissue and to address possible leakage/backbleed issues. These methods include using high-dose bovine thrombin at 1000 U/mL to make APG. An agitator mechanism can be used in the PRP delivery and/or refill chamber to prevent settling or dissolution of the PRP. This will ensure delivery of a homogeneous PRP to the target tissue and facilitate improved clotting. Other methods include allowing the needle to dwell for 5-10 second in the injection site after the injectate has been delivered, using an oblique angle to lengthen the injection track in the tissue, and local stabilization of the injection site on entry of the needle (to prevent tearing). Each of these methods was tested in the aforementioned Examples.

Using cooled (˜0° C.) thrombin to make APG also enhances retention of the injectate in the target tissue. For the embodiment using cooled thrombin, a refrigerated/cooled chamber can be used in the thrombin delivery and/or refill chamber.

The injected compositions can be visualized by intra-operative ECHO (echocardiography), which can be used to confirm adequate needle placement and retention. The ECHO can be used as a separate device or can be included within the delivery system (e.g. similar to intravascular ultrasound [IVUS]).

Unintended Perforation of a heart chamber and/or delivery into chamber blood (or blood vessels), can be avoided by using imaging guidance during injections, such as that provided by ECHO or IVUS. Additionally, it was found that direct epicardial injections into the apex of the heart should be avoided to prevent chamber puncture. Instead, oblique injections should be used to access apical tissue. Also, a device can be used to inform the operator when the delivery portion of the delivery device is in an undesired position for delivery, such as in the ventricle or in a coronary vessel. Such a device may have at least one sensor include, but not limited to, a pressure sensor, a color detector, an oxygen sensor, a carbon dioxide sensor or a lumen to express backflowing blood under pressure that generates a unique signal when the delivery system is positioned such that its target is in a blood space. Once alerted, the user can re-position the device before delivering the composition.

These experiments have shown that the methods disclosed herein can be used to restore infarct left ventricular wall thickness to (or beyond) pre-MI levels immediately following injections. This favorable effect persists (reproducibly) out to 1 week. The methods can also restore left ventricular ejection fraction (EF) to pre-MI levels immediately following injections. Additionally, treatments disclosed herein can improve cardiac dynamics and function post-MI by giving dyskinetic segments of left ventricular tissue akinetic properties.

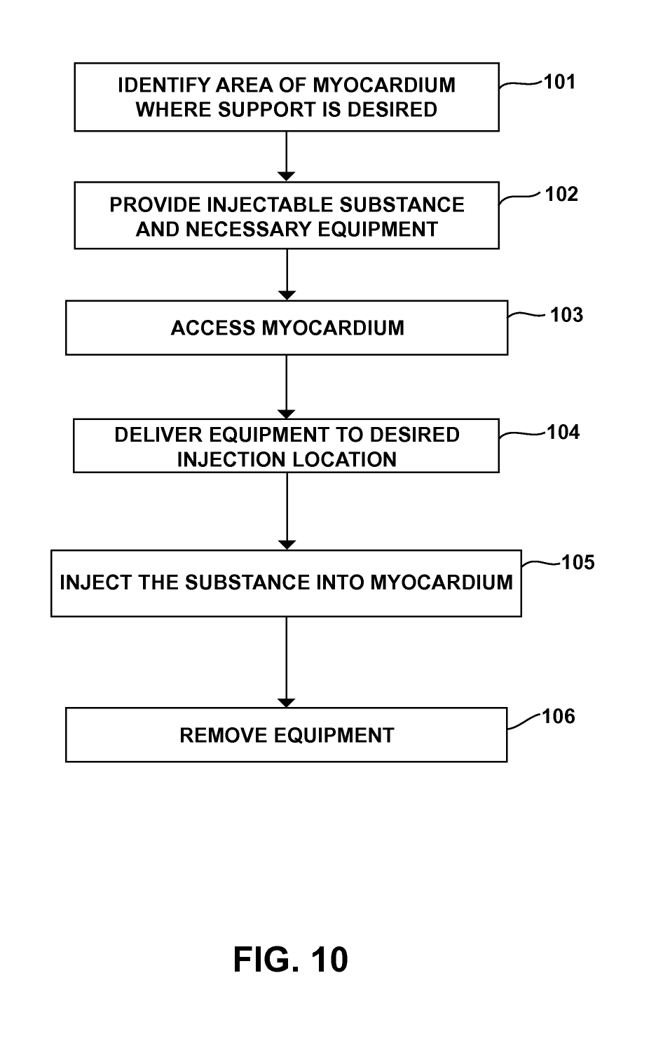

The current invention discloses a method of treating ischemic myocardium by injecting substances that provide structural reinforcement of the tissue or structural reinforcement of the tissue in conjunction with biological therapy. Referring to FIG. 10, the method generally comprises the steps of identifying and/or imaging the ischemic region of myocardium where support is desired 101, determining an appropriate substance for injecting into the myocardium to achieve the desired effect (structural reinforcement of the tissue or structural reinforcement of the tissue combined with biological therapy) and selecting the appropriate device for injecting the substance into the myocardium 102, accessing the myocardium 103, delivering the substance and delivery device to the desired treatment location 104, injecting the substance into the myocardium 105 and withdrawing the device 106. The method and devices for injecting the composition (substance/injectate), the composition, and the processes for delivery have been discussed herein.

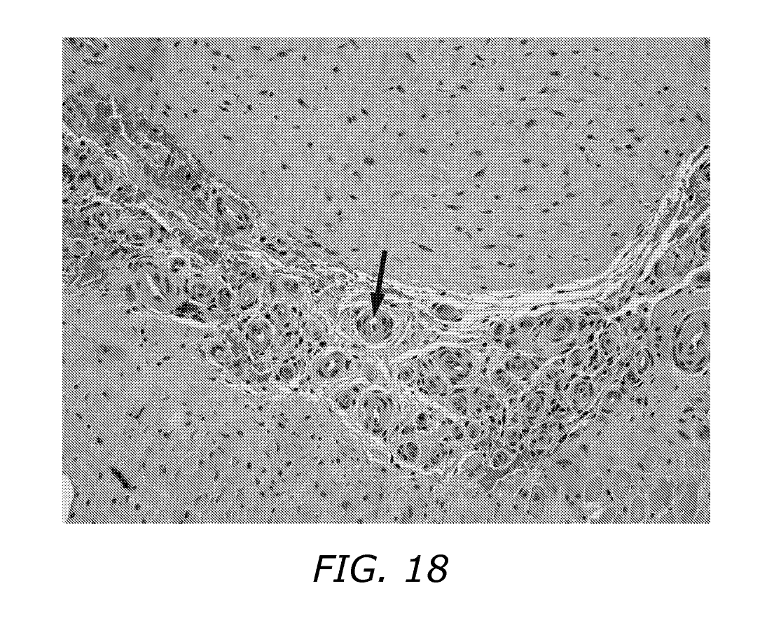

Furthermore, as seen in FIG. 17, the system of the current invention comprises identification of the injured area of myocardium and the treatment site, accessing the treatment site with a delivery device, injecting the composition at one or more locations at the treatment site in the myocardium and removing the delivery device from the patient.

It will be appreciated by those skilled in the art that while the present invention has been described above in connection with particular embodiments and examples, the invention is not necessarily so limited, and that numerous other embodiments, examples, uses, modifications and departures from the embodiments, examples and uses are intended to be encompassed by the claims attached hereto. The entire disclosure of each patent and publication cited herein is incorporated by reference, as if each such patent or publication were individually incorporated by reference herein.

What is claimed is:

1. A system for preventing chamber remodeling of an injured heart by structurally reinforcing the cardiac tissue comprising:
at least one composition; and
at least one delivery device for introducing said composition into said cardiac tissue;
wherein said composition provides structural support for said cardiac tissue.

2. The system of claim 1 further comprising a cardiac stabilization device.

3. The system of claim 1 further comprising an imaging device.

4. The system of claim 3 wherein said imaging device is an echocardiography device.

5. The system of claim 1 wherein said composition comprises one or more than one structural material selected from the group consisting of platelet gel, autologous platelet gel, collagen, biocompatible polymers, alginates, synthetic/natural compounds, fibrinogen, silk-elastin polymers, hydrogels, and dental composite material.

6. The system of claim 1 wherein said composition is delivered to said treatment site and forms a solid or a gel within said cardiac tissue at said treatment site.

7. The system of claim 6 wherein said composition forms a solid or a gel as a result of physical or chemical cross-linking or activation, wherein said activation is selected from the group consisting of enzymatic, chemical, thermal of light activation of said composition.

8. The system of claim 5 wherein said composition comprises autologous platelet gel.

9. The system of claim 8 wherein said autologous platelet gel is formed from platelet poor plasma or platelet rich plasma and an activating agent.

10. The system of claim 9 wherein said activating agent is thrombin.

11. The system of claim 10 wherein said thrombin is selected from the group consisting of recombinant thrombin, human thrombin, animal thrombin, engineered thrombin and autologous thrombin.

12. The system of claim 1 wherein said at least one composition comprises two or more compositions and said two or more compositions are injected approximately simultaneously at said treatment site.

13. The system of claim 1 wherein said composition further comprises a bioactive agent.

14. The system of claim 13 wherein said bioactive agent is selected from the group consisting of pharmaceutically active compounds, hormones, growth factors, enzymes, DNA, RNA, siRNA, viruses, proteins, lipids, polymers, hyaluronic acid, antibodies, antibiotics, anti-inflammatory agents, anti-sense nucleotides and transforming nucleic acids, and combinations thereof.

15. The system of claim 1 wherein said composition further comprises a contrast agent.

16. The system of claim 8 wherein said composition further comprises an agent to increase the structural strength of said composition.

17. The system of claim 16 wherein said agent to increase the structural strength of said composition is fibrinogen.

18. The method of claim 1 wherein said composition is provided to said injured cardiac tissue between 1 hour and 2 weeks after injury occurs to said cardiac tissue.

19. The system of claim 1 wherein said composition is provided in approximately 1 to 20 injections.

20. The system of claim 19 wherein said injections are provided sequentially.

21. The system of claim 19 wherein said injections are provided approximately simultaneously.

22. The system of claim 19 wherein said composition comprises a total injection volume up to 15 mL.

23. The system of claim 19 wherein said composition comprises an injection volume up to 1100 microliters per injection.

24. The system of claim 1 wherein said composition is injected into said cardiac tissue at an angle orthogonal or oblique to the tissue surface.

25. The system of claim 1 wherein said injection site in said cardiac tissue is selected from the group consisting of sub-endocardial, sub-epicardial and intra-myocardial sites.

26. The system of claim 25 wherein said composition is injected into said cardiac tissue at a depth midway through the thickness of the myocardium.

27. The system of claim 10 wherein said ratio of platelet rich plasma or said platelet poor plasma to said thrombin is between approximately 5:1 to approximately 25:1.

28. The system of claim 27 wherein said ratio of platelet rich plasma or said platelet poor plasma to said thrombin is approximately 10:1.

29. The system of claim 1 further comprising a delivery device adapted to deliver said composition into said injured cardiac tissue.

30. The system of claim 29 wherein said delivery device is an injection catheter selected from the group consisting of an endocardial injection catheter, a transvascular injection catheter and an epicardial injection catheter.

31. The system of claim 1 wherein said composition is provided to said treatment site during an injurious event or after an injurious event has occurred.

32. The system of claim 1 wherein said treatment site is selected from the group consisting of the injured area, the peri-injury area and the healthy tissue surrounding the injured area.

33. A system for preventing chamber remodeling of an injured heart by structurally reinforcing the cardiac tissue comprising:
autologous platelet gel wherein said autologous platelet gel is comprised of platelet rich plasma and thrombin in a ration of 10:1.
at least one delivery device for introducing said autologous platelet into said cardiac tissue; and
wherein said autologous platelet gel provides structural support for said cardiac tissue.
